(19) United States
(12) Patent Application Publication
(10) Pub. No.: US 2008/0193330 A1
(43) Pub. Date: Aug. 14, 2008

(54) surface treatment apparatus

(75) Inventors: Eiki Hotta, Yokohama-shi (JP); Naohiro Shimizu, Miura-shi (JP); Yuichiro Imanishi, Nagoya-shi (JP)

Correspondence Address: OLIFF & BERRIDGE, PLC, P.O. BOX 320850, ALEXANDRIA, VA, 22320-4850, US

(73) Assignees: TOKYO INSTITUTE OF TECHNOLOGY, TOKYO (JP); NGK INSULATORS, LTD., NAGOYA-SHI (JP)

(21) Appl. No.: 11/826,957

(22) Filed: Jul. 19, 2007

(30) Foreign Application Priority Data
Feb. 9, 2007 (JP) 2007-031297
Mar. 16, 2007 (JP) 2007-068908

(51) Int. Cl. A61L 2/14

(52) U.S. Cl. 422/23

(57) ABSTRACT

A surface treatment apparatus encompasses a gas introducing system configured to introduce a process gas from one end of a tubular treatment object; a vacuum evacuating system configured to evacuate the process gas from other end of the treatment object; an excited particle supplying system disposed at the gas supply upstream side to the treatment object, configured to supply excited particles for inducing initial discharge in a main body of the treatment object; and a first main electrode and a second main electrode disposed oppositely to each other, defining a treating region of the treatment object as a main plasma generating region disposed therebetween, wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the treatment object, and thereby an inner surface of the treatment object is treated.

### CROSS REFERENCE TO RELATED APPLICATIONS AND INCORPORATION BY REFERENCE

This application claims benefit of priority under 85 USC 119 based on Japanese Patent Application No. P2007-31297 filed Feb. 9, 2007, and Japanese Patent Application No. P2007-68908 filed Mar. 16, 2007, the entire contents of which are incorporated by reference herein.

### BACKGROUND OF INVENTION

1. Field of Invention

The present invention pertains to a surface treatment apparatus using non-thermal equilibrium low temperature plasma. Invention particularly relates to a surface treatment apparatus that facilitates miscellaneous inner wall processing of treatment objects, which may include a long (several meters long) and narrow (several millimeters of inside diameter) dielectric tube.

2. Description of the Related Art

Liquid in a narrow tube contact with inner wall of the narrow tube at a specific contact angle, the value of the contact angle depends upon surface property of inner wall such as hydrophobic or hydrophilic behavior and geometry of inner wall such as glassy shape or hollow shape. An upward force in a pipe of a capillary action depends on the product of surface tension, cosine of a contact angle, and circumferential length of a hole. A downward force depends on the product of pressure, gravity, specific gravity of the liquid and height of the liquid. Therefore, the height of the liquid in a narrow tube can be calculated by equating the upward force and the downward force. For example, a column of water rises about 0.75 m in an atmospheric pressure in a pipe element having an inside diameter of 20 micrometers. However, in the inner wall of a narrow tube, it is difficult that liquid is transported at high speed. Therefore, as against inside of a long-narrow tube, it is extremely difficult to execute pasteurization, sterilization or washing by wet processing. Because of these problems, dry-process is suitable for inner wall processing of a long-narrow tube by non-thermal equilibrium low temperature plasma, which is full of radicals, is expected to process inner wall of a narrow tube.

Ichiki et al. have proposed an employment of plasma jet generated by inductively-coupled-high-frequency plasma for the dry-process of inner wall of a narrow tube is tried (See T. Ichiki et al., “Localized and ultrahigh-rate etching of silicon wafers using atmospheric-pressure microplasma jet”, J. Appl. Phys., 95 (2004) pp. 35-39). Plasma length of Ichiki et al. is around several centimeters to the utmost.

Fujiyama proposed a configuration in which a metal electrode is interposed in a narrow tube so as to establish a pulsed discharge. However, it is extremely difficult to interpose the metal electrode in inside of a narrow tube having an inside diameter of less than several millimeters (See H. Fujiyama, “Inner coating of long-narrow tube by plasma sputtering”, Surface and Coating Technology, 131 (2000) pp. 278-283).

In particular, because medical instrument such as endoscope encompasses optical system and metallic parts having very minute geometry, the metallic part rises to a considerable high temperature, when the medical instrument are sterilized by plasma, even though low temperature plasma is employed. The rising to the high temperature generates a problem that warp or misalignment is produced m the optical system.

Because of these problems, under the present situations, in order to remove microbes adhered to an endoscope, a medical staff must dip the endoscope in antiseptic solution, and wash off microbes carefully from the endoscope with several stages in the antiseptic solution.

In view of these situations, Fukuda has proposed another sterilization method in a double tube structure, establishing washing in water and sterilization by plasma (See JP2006-21027 A). A long-narrow tube to be sterilized is dipped into water, which is filled in an inner tube made of glass, and the inner tube is installed man outer tube. The plasma generated in a space between the inner tube and the outer tube is irradiated to long-narrow tube through the inner tube. However, in the double tube method proposed by Fukuda, because a basis of sterilization is wet processing, there is a limit in the sterilization capability.

Therefore, no effective plasma generation method is proposed, which can be applied to in the inside of a long-narrow tube, having a length of several meters and an inside diameter of several millimeters, until now.

In particular, because dissociation energy of nitrogen molecules is so large compared with other gas molecules, as shown in table 1, as for the generation of nitrogen plasma, stable generation was very difficult until now.

TABLE 1

| | gas molecules | | | | | | | |
|---|---|---|---|---|---|---|---|---|
| | F₂ | H₂O₂ | OH | N₂O | O₂ | CO₂ | NO | N₂ |
| dissociation | 1.66 | 2.21 | 4.62 | 4.93 | 5.21 | 5.52 | 6.50 | 9.91 |
| energy (eV) | | | | | | | | |

### SUMMARY OF INVENTION

In view of these situations, it is an object of the present invention to provide a surface treatment apparatus, which can treat surfaces of inner walls of various kinds of treatment objects, including a long-narrow tube having a length of several meters with an inside diameter of several millimeters. Hereinafter, the term “inner wall treatment” shall mean any surface treatment of a surface of inner wall of the subject treatment object. In addition, the term “surface treatment” shall mean any surface treatment of a surface of inner wall (inner surface) or the outer wall (outer surface) of the subject treatment object, which may include pasteurization, sterilization, and improvement of wettability. In a wide sense, the term “surface treatment” shall mean any removal of adhered materials, such as organic/inorganic materials, adhered to the surface of inner wall (inner surface) or the outer wall (outer surface) of the treatment object and any change of physical or chemical property of inner surface or the outer surface of the treatment object.

The term “change of physical or chemical property” shall include deposition or etching by plasma reaction. Therefore, a process to deposit a film made of material different from inner surface of the treatment object corresponds to the term “change of physical or chemical property”.

An aspect of the present invention inheres in a surface treatment apparatus encompassing a gas introducing system for introducing a process gas from one end of a tubular treatment object a vacuum evacuating system for evacuating the process gas from other end of the treatment object; an excited particle supplying system disposed at the gas supply upstream side to the treatment object, for supplying excited particles for inducing initial discharge in a main body of the treatment object and a first main electrode and a second main electrode disposed oppositely to each other, defining a treating region of the treatment object as a main plasma generating region disposed therebetween, wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the treatment object, and thereby the inner surface of the treatment object is treated.

Another aspect of the present invention inheres in a surface treatment apparatus encompassing a vacuum evacuating system for evacuating a process gas introduced at a specific flow rate from an introducing piping provided at other end of a tubular treatment object having one end closed, from an exhaust piping provided at the other end, and maintaining the pressure of the process gas inside the treatment object at a process pressure; an excited particle supplying system disposed at the gas supply upstream side to the treatment object, for supplying excited particles for inducing initial discharge in a main body of the treatment object; and a first main electrode and a second main electrode disposed oppositely to each other, defining a treating region of the treatment object as a main plasma generating region disposed therebetween, wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the treatment object, and thereby the inner surface of the treatment object is treated.

Still another aspect of the present invention inheres in a surface treatment apparatus encompassing a vacuum manifold unit connected to other end of a tubular treatment object having one end closed, for sealing process gas at specified pressure inside of the treatment object from the other end; an excited particle supplying system disposed at the other end side, for supplying excited particles for inducing initial discharge in a main body of the treatment object; and a first main electrode and a second main electrode disposed oppositely to each other, defining a treating region of the treatment object as a main plasma generating region disposed therebetween, wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the treatment object, and thereby the inner surface of the treatment object is treated.

Further aspect of the present invention inheres in a surface treatment apparatus encompassing a vacuum evacuating system for generating a gas flow by evacuating a process gas introduced from one end of a tubular trunk pipe of a treatment object, the treatment object having the tubular trunk pipe and a branch pipe branched off from the trunk pipe, from the other end of the trunk pipe and an end portion of the branch pipe; an excited particle supplying system disposed at the gas supply upstream side to the treatment object, for supplying excited particles for inducing initial discharge in a main body of the treatment object; and a first main electrode and a second main electrode disposed oppositely to each other, defining a treating region of the treatment object as a main plasma generating region disposed therebetween, wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the treatment object, and thereby the inner surface of the treatment object is treated.

Still further aspect of the present invention inheres in a surface treatment apparatus encompassing a vacuum evacuating system for generating a gas flow by evacuating a process gas introduced from one end of a tubular trunk pipe of a treatment object and end portion of a branch pipe of the treatment object, the treatment object having the tubular trunk pipe and the branch pipe branched off from the trunk pipe, from the other end of the trunk pipe; an excited particle supplying system disposed at the gas supply upstream side to the treatment object, for supplying excited particles for inducing initial discharge in a main body of the treatment object; and a first main electrode and a second main electrode disposed oppositely to each other, defining a treating region of the treatment object as a main plasma generating region disposed therebetween, wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the treatment object, and thereby the inner surface of the treatment object is treated.

Still further aspect of the present invention inheres in a surface treatment apparatus encompassing an excited particle supplying system disposed at the gas supply upstream side of a tubular treatment object made of dielectric material, the treatment object having a length greater than the diameter, for supplying excited particles for inducing initial discharge in a main body of the treatment object; and a first main electrode and a second main electrode disposed oppositely to each other, defining a treating region of the treatment object as a main plasma generating region disposed therebetween, wherein a process gas is introduced from one end of the treatment object to form a gas flow inside of the treatment object, and the pressure of the gas flow is adjusted to a process pressure in a range of 20 kPa to 100 kPa, the excited particle supplying system is driven at least until generation of main plasma, and main pulse of duty ratio of 10⁻⁷ to 10⁻¹ is applied between the first main electrode and second main electrode to generate a non-thermal equilibrium plasma flow inside the treatment object, and thereby the inner surface of the treatment object is treated.

Still further aspect of the present invention inheres in a surface treatment apparatus encompassing a dielectric housing configured to accommodate an treatment object; a gas introducing system configured to introduce a process gas from one end of the dielectric housing; a vacuum evacuating system configured to evacuate the process gas from other end of the dielectric housing; an excited particle supplying system disposed at the gas supply upstream side to the dielectric housing, configured to supply excited particles for inducing initial discharge in a main body of the dielectric housing; and a first main electrode and a second main electrode disposed oppositely to each other, defining a treating region of the treatment object as a main plasma generating region disposed therebetween, wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the dielectric housing, and thereby a surface of the treatment object is treated.

Still further aspect of the present invention inheres in a surface treatment apparatus encompassing a dielectric housing configured to accommodate an treatment object; a vacuum evacuating system configured to evacuate a process gas introduced at a specific flow rate from an introducing piping provided at other end of the dielectric housing having one end closed, from an exhaust piping provided at the other end, and maintaining the pressure of the process gas inside the dielectric housing at a process pressure; an excited particle supplying system disposed at the gas supply upstream side to the dielectric housing, configured to supply excited particles for inducing initial discharge in a main body of the dielectric housing; and a first main electrode and a second main electrode disposed oppositely to each other, defining a treating region of the treatment object as a main plasma generating region disposed therebetween, wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the dielectric housing, and thereby a surface of the treatment object is treated.

Other and further objects and features of the present invention will become obvious upon an understanding of the illustrative embodiments about to be described in connection with the accompanying drawings or will be indicated in the appended claims, and various advantages not referred to herein will occur to one skilled in the art upon employing of the present invention in practice.

### BRIEF DESCRIPTION OF THE DRAWINGS

Various embodiments of the present invention will be described with reference to the accompanying drawings. It is to be noted that the same or similar reference numerals are applied to the same or similar parts and elements throughout the drawings, and the description of the same or similar parts and elements will be omitted or simplified. Generally and as it is conventional in the representation of semiconductor devices, it will be appreciated that the various drawings are not drawn to scale from one figure to another nor inside a given figure, and in particular that the layer thicknesses are arbitrarily drawn for facilitating the reading of the drawings.

FIG. 1 is a schematic diagram explaining the principle of a surface treatment apparatus in accordance with a first embodiment of the present invention;

FIG. 2 is a bird's-eye view specifically explaining part of the surface treatment apparatus in accordance with the first embodiment of the present invention;

FIG. 3A is a bird's-eye view explaining a meandering treatment object guide groove for accommodating a flexible long-narrow tube adapted for the surface treatment apparatus in accordance with the first embodiment of the present invention;

FIG. 3B is a schematic sectional view explaining an accommodated state of the treatment object in the treatment object guide grooves shown in FIG. 3A;

FIG. 4A shows a voltage waveform of high voltage pulse applied between a first main electrode and a second main electrode in the surface treatment apparatus in accordance with the first embodiment of the present invention;

FIG. 4B shows a corresponding current waveform to the voltage waveform of high voltage pulse shown in FIG. 4A;

FIG. 5 is a schematic diagram explaining an electric field distribution, when a treatment object made of dielectric material is disposed in parallel between first main electrode and second main electrode implementing a parallel flat electrode configuration;

FIG. 6 is a sectional diagram schematically explaining essential structure of a surface treatment apparatus in accordance with a second embodiment of the present invention;

FIG. 7 is a schematic plan view explaining a configuration of a plurality of gas supply holes of the surface treatment apparatus in accordance with the second embodiment of the present invention;

FIG. 8 is a cross-sectional view schematically explaining essential structure of a surface treatment apparatus in accordance with a first modification of the second embodiment of the present invention;

FIG. 9 is a cross-sectional view schematically explaining essential structure of a surface treatment apparatus in accordance with a second modification of the second embodiment of the present invention;

FIG. 10 is a cross-sectional view schematically explaining essential structure of a surface treatment apparatus in accordance with a third embodiment of the present invention;

FIG. 11 is a cross-sectional view schematically explaining essential structure of a surface treatment apparatus in accordance with a fourth embodiment of the present invention;

FIG. 12 is a cross-sectional view schematically explaining essential structure of a surface treatment apparatus in accordance with a fifth embodiment of the present invention;

FIG. 13 is a cross-sectional view schematically explaining essential structure of a surface treatment apparatus in accordance with a first modification of the fifth embodiment of the present invention;

FIG. 14 is a cross-sectional view schematically explaining essential structure of a surface treatment apparatus in accordance with a second modification of the fifth embodiment of the present invention;

FIG. 15 is a cross-sectional view schematically explaining essential structure of a surface treatment apparatus in accordance with a sixth embodiment of the present invention;

FIG. 16 is a cross-sectional view schematically explaining essential structure of a surface treatment apparatus in accordance with a seventh embodiment of the present invention;

FIG. 17 is a cross-sectional view schematically explaining essential structure of the surface treatment apparatus in accordance with the seventh embodiment as seen from a direction orthogonal to FIG. 16;

FIG. 18 is a cross-sectional view schematically explaining essential structure of a surface treatment apparatus in accordance with an eighth embodiment of the present invention;

FIG. 19 is a cross-sectional view schematically explaining essential structure of the surface treatment apparatus in accordance with the eighth embodiment as seen from a direction orthogonal to FIG. 18;

FIG. 20 is a cross-sectional view schematically explaining essential structure of a surface treatment apparatus in accordance with a ninth embodiment of the present invention;

FIG. 21 is a cross-sectional view schematically explaining essential structure of a surface treatment apparatus in accordance with a tenth embodiment of the present invention;

FIG. 22 is a cross-sectional view schematically explaining essential structure of a surface treatment apparatus in accordance with an eleventh embodiment of the present invention;

FIG. 23 is a cross-sectional view schematically explaining essential structure of a surface treatment apparatus in accordance with a first modification of the eleventh embodiment of the present invention;

FIG. 24 is a cross-sectional view schematically explaining essential structure of a surface treatment apparatus in accordance with a second modification of the eleventh embodiment of the present invention;

FIG. 25 is a cross-sectional view schematically explaining essential structure of a surface treatment apparatus in accordance with a third modification of eleventh embodiment of the present invention;

FIG. 26 is a cross-sectional view schematically explaining essential structure of a surface treatment apparatus in accordance with a fourth modification of the eleventh embodiment of the present invention;

FIG. 27 is a cross-sectional view schematically explaining essential structure of a surface treatment apparatus in accordance with a twelfth embodiment of the present invention;

FIG. 28A illustrates an example of dielectric triple points, which can be employed in the surface treatment apparatus in accordance with the twelfth embodiment of the present invention;

FIG. 28B illustrates another example of dielectric triple points, which can be employed in the surface treatment apparatus in accordance with the twelfth embodiment of the present invention;

FIG. 29 is a cross-sectional view schematically illustrating a treatment object under treatment by a surface treatment apparatus in accordance with a thirteenth embodiment of the present invention;

FIG. 30 illustrates Paschen's law, which serves as a basis of the surface treatment apparatus of the thirteenth embodiment of the present invention;

FIG. 31 is a cross-sectional view schematically illustrating a state when the treatment against the treatment object is completed in the surface treatment apparatus of the thirteenth embodiment of the present invention;

FIG. 32 is a cross-sectional view schematically illustrating another state when the treatment against the treatment object is completed in the surface treatment apparatus of the thirteenth embodiment of the present invention;

FIG. 33 is a cross-sectional view schematically illustrating a treatment object under treatment by a surface treatment apparatus in accordance with a modification of the thirteenth embodiment of the present invention;

FIG. 34 is a cross-sectional view schematically illustrating a state when the treatment against the treatment object is completed in the surface treatment apparatus of the modification of the thirteenth embodiment of the present invention;

FIG. 35 is a cross-sectional view schematically illustrating another state when the treatment against the treatment object is completed in the surface treatment apparatus of the modification of the thirteenth embodiment of the present invention;

FIG. 36A is a cross-sectional view cut along axial direction, schematically explaining structure of an excited particle supplying system of a surface treatment apparatus in accordance with another embodiment of the present invention;

FIG. 36B is a corresponding cross-sectional view cut along radial direction of the excited particle supplying system shown in FIG. 36A;

FIG. 37A is a cross-sectional view cut along axial direction, schematically explaining structure of another excited particle supplying system of a surface treatment apparatus in accordance with another embodiment of the present invention; and

FIG. 37B is a corresponding cross-sectional view cut along radial direction of the excited particle supplying system shown in FIG. 37A.

### DETAILED DESCRIPTION OF INVENTION

In the following description specific details are set forth, such as specific materials, processes and equipment in order to provide a thorough understanding of the present invention. It will be apparent, however, to one skilled in the art that the present invention may be practiced without these specific details. In other in stances, well-known manufacturing materials, processes and equipment are not bet forth in detail in order not to unnecessarily obscure the present invention. Prepositions, such as “on”, “over”, “under”, “beneath”, and “normal” are defined with respect to a planar surface of the object component, regardless of the orientation in which the object component is actually held. A layer is on another layer even if there are intervening layers.

### First Embodiment

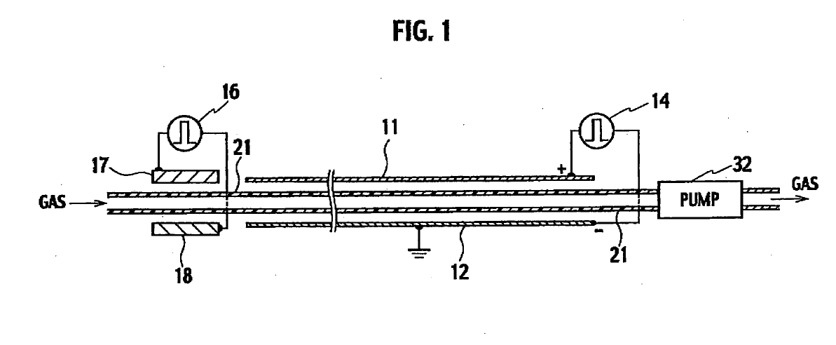

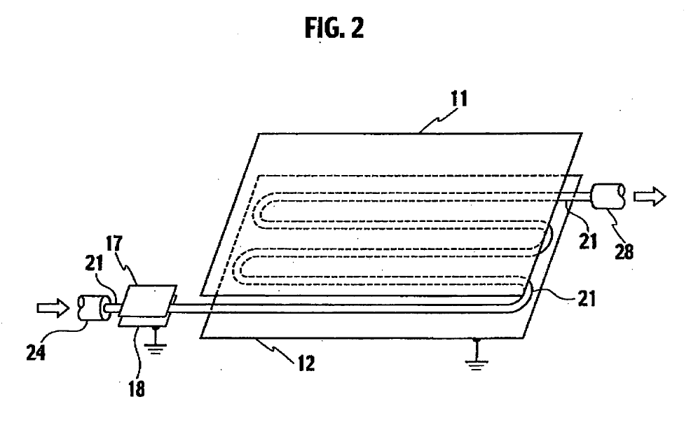

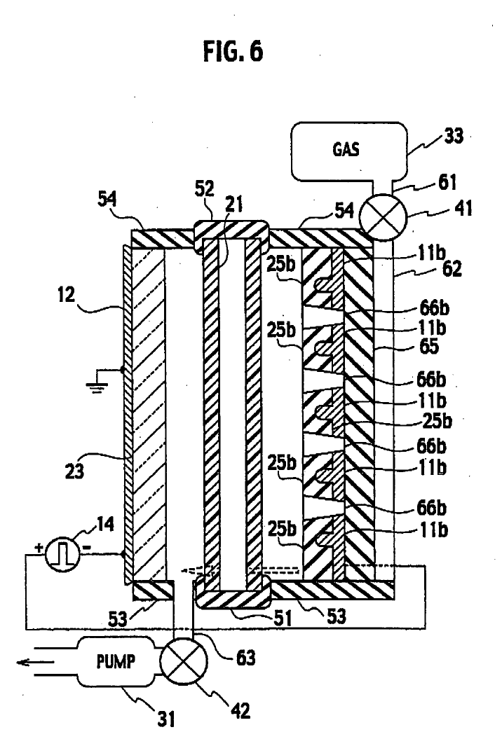

As shown in FIGS. 1 and 2, a surface treatment apparatus related to a first embodiment of the present invention encompasses a gas introducing system (illustration is omitted, but the gas introducing system is shown in FIG. 6) for introducing a process gas from one end of a tubular treatment object 21; a vacuum evacuating system 32 for evacuating the process gas from other end of the treatment object 21; an excited particle supplying system (16, 17 and 18) disposed at the gas supply upstream side to the treatment object 21, for supplying excited particles for inducing initial discharge in a main body of the treatment object 21; and a first main electrode 11 and a second main electrode 12 disposed oppositely to each other, defining a treating region of the treatment object 21 as a main plasma generating region disposed therebetween, wherein the excited particle supplying system (16, 17 and 18) is driven at least until generation of main plasma, and main pulse of duty ratio of 10⁻⁷ to 10⁻¹ is applied between the first main electrode 11 and second main electrode 12, to generate a non-thermal equilibrium plasma now inside the treatment object 21, and thereby the inner surface of the treatment object 21 is treated.

In FIGS. 1 and 2, the second main electrode (cathode electrode) 12 that is illustrated at lower side is grounded, while to the first main electrode (anode electrode) 11 that is frustrated at upper side is illustrated, a high voltage is applied. But the drawing is illustrative, and top and bottom relation of a drawing, or right and left relation of the drawing can be defined and expressed arbitrary. For example, the second main electrode 12 illustrated at lower side can be assigned as anode electrode, while the first main electrode 11 illustrated at upper side can be assigned as cathode electrode, theoretically. If the second main electrode 12 is kept to be grounded, the polarity of the output pulse of the power supply 14 is reversed so that the first main electrode 11 can serve as the cathode. On the other hand, the first main electrode 11 can be grounded without turning over the polarity of the output pulse of the power supply 14 such that a high voltage is applied to the second main electrode 12, the first main electrode 11 can serve as the cathode electrode.

The technical feature such that, in a surface treatment apparatus related to the first embodiment, a long-narrow tube having an inside diameter of less than or equal to 7-5 millimeters and a length of more than 4-7 meters is supposed to be employed as the treatment object 21 having tubular geometry, but even if the length is equal to or less than 4 meters long or inside diameter is more 7 millimeters, the treatment object 21 can be processed, may be understood from the following discussion.

In particular, as for the technical advantage of the surface treatment apparatus related to the first embodiment, because, in Ichiki's methodology, the length of a microplasma is several centimeters at longest, a tube having a length of around 10 centimeters can achieve a significant effectiveness over Ichiki's methodology. In view of the technology taught by Ichiki's methodology, in a technical field of plasma, a tube having an inside diameter of equal to or less than 7-5 millimeters, a length of more than around 10 centimeters can be defined as “a long-narrow tube”. In addition, a cross-section of treatment object 21 is not limited to a circle, but polygons, including rectangle, can be employed. However, as for the long-narrow tubes adapted for industrial applications, there will be many cases that the long-narrow tubes have a circular cross-section. Although as representative long-narrow tube, medical instrument such as an endoscope (fiber scope) is well known, the technical concept of “a long-narrow tube” covers through various kinds of narrow tubes. For example, narrow tubes adapted for drinking water, which is used in vending machines can be included in the technical concept of “a long-narrow tube”.

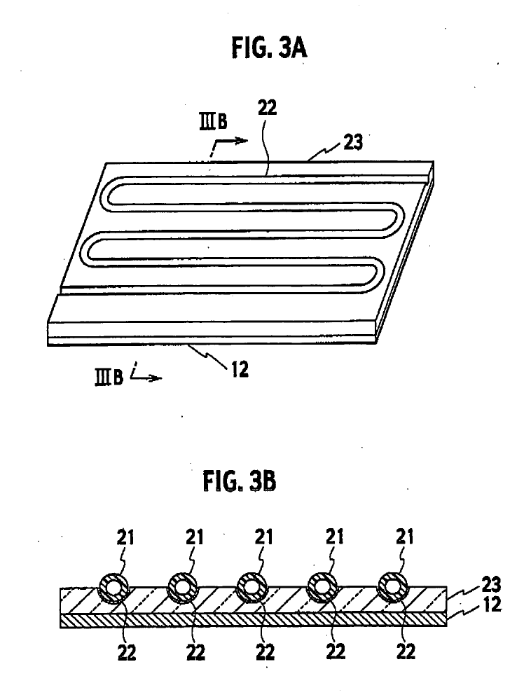

When the treatment object 21 is a flexible long-narrow tube having an inside diameter equal to or less than around several millimeters, and a length of more than around several meters, and further the length is known beforehand, as shown in FIGS. 3A and 3B, a second main, electrode covering insulator 23 of high purity quartz glasses is provided on the second main electrode 12, such that a meandering treatment object guide groove 22 is cut in and at the surface of second main electrode covering insulator 23. Then, the flexible long-narrow tube can be fixed in the treatment object guide groove 22, by bending at one corner or plural number of corners, the number of corners depends on the length of the flexible long-narrow tube as shown in FIG. 3B. Because the configuration of the treatment object guide groove 22 can be designed so as to conform to the length of the treatment object 21, if each of the lengths of the treatment objects 21 are predetermined, like a case of medical instrument, a plurality of second main electrode covering insulators 23, each having different length of treatment object guide groove 22 corresponding to the length of the treatment objects 21 may be prepared.

Anyhow, the configuration with the treatment object guide groove 22 shown in FIGS. 3A and 3B is a mere example, and various kinds of structure can be adopted, in fact. For example, a hook structure implemented by a plurality of protrusions or a screw structure having a plurality of screws may be established on a top surface of the flat second main electrode covering insulator 23 so as to fix the treatment object 21 with a plurality affixing sites.

If the treatment object 21 is the flexible long-narrow tube, rather than the configuration shown in FIGS. 3A and 3B, first and second reels may be provided so that one end of the treatment object 21 can be rolled up by the first reel, while the second reel provided at another end of the treatment object 21 rolls out the treatment object 21, thereby establishing internal surface treatment of the treatment object 21 may be conducted partially and sequentially. Therefore, it is illustrated as if the full length of the treatment object 21 and the length of the first main electrode 11 and the second main electrode 12 are approximately equal in FIG. 1, but depending on behaviors of material such as flexure property, expansive property and contractive property of the treatment object 21, the relationship between the full length of the treatment object 21 and each length of the first main electrode 11 and the second main electrode 12 can be elected arbitrary.

The excited particle supplying system (16, 17 and 18) encompasses a first auxiliary electrode 17, a second auxiliary electrode 18 facing to the first auxiliary electrode 17 so as to sandwich the upper stream side of the treatment object 21, implementing a configuration of a parallel plate electrode, and an auxiliary pulse power supply 16 configured to supply electric pulses between the first auxiliary electrode 17 and the second auxiliary electrode 18. The excited particle supplying system (16, 17 and 18) is provided so as to the starting voltage of the discharges and to generate initial plasma so as to facilitate generation of the plasma in the treatment object 21.

In addition to the effect such that generated plasma or excited particle are transported by diffusion and flow of process gas to arrive in the inside of the treatment object 21, an effect of irradiation by the light emitted from the generated plasma in the excited particle supplying system (16, 17 and 18) can be expected so that light can ionize neutral particles in the treatment object 21. Once plasma is generated in the treatment object 21, and if density of charged particles is large enough, an discharge is realized in the treatment object 21 only by the electric field established between the first main electrode 11 and the second main electrode 12, and the generated plasma can be maintained in the treatment object 21. In this stage, the excited particle supplying system (16, 17 and 18) is not needed any more. Therefore, the excited particle supplying system (16, 17 and 18) is employed only at the initial stage of the plasma generation.

In addition, because it is enough that initial plasma can be injected in the flow of gas in the early stage, the excited particle supplying system may be implemented by any other configuration such as an inductive plasma source which can generate initial plasma, and the excited particle supplying system is not limited to the parallel plate electrode configuration shown in FIG. 1.

After excitation of initial plasma, the surface treatment apparatus shown in FIG. 1 execute treatment in the inside of the treatment object 21 by radicals included in the plasma generated by discharge. In the surface treatment apparatus related to the first embodiment, high purity nitrogen gas is supplied as process gas in the treatment object 21 from the upper-stream side, but “process gas” is not always limited to nitrogen gas. For example, for pasteurization or sterilization of inside of the treatment object 21, even mixed gas of chlorine (Cl₂) gas or compound gas including chlorine, or more generally, various kinds of active gas such as halogen based compound gas, or mixed gas of these active gas with nitrogen gas can be employed. Even oxygen (O₂) gas or various compound gas including oxygen is available, depending on the object of the surface treatment. The purity or the dew point of the process gas may be determined appropriately in view of the object of surface treatment.

In the surface treatment apparatus related to the first embodiment, the process gas is supplied in the treatment object 21 as shown in FIG. 1 from the upper-stream aide, and the process gas flows through the treatment object 21, and the treatment object 21 is kept at a processing pressure of less than or equal to an atmospheric pressure by vacuum pump 32 arranged downstream. Although the illustration is omitted in FIG. 1, a pressure gauge and a variable conductance valve configured to adjust the exhaust conductance maybe provided, as a person skilled in the art may easily understand. For example, a pressure gauge and a mass-flow controller configured to control the flow rate are provided to intake adapter 24 as shown in FIG. 2, and a variable conductance valve adjusting the exhaust conductance maybe established in the exhaust adapter 28 shown in FIG. 2. In addition, a pressure gauge may be provided to the exhaust adapter 28.

Intake adapter 24 shown in FIG. 2 is a piping including a vacuum tight connection joint, configured to connect the supply of process gas such as gas cylinder, illustration of which is omitted, and one end of the treatment object 21. The exhaust adapter 28 is another piping including a vacuum tight connection joint configured to connect the vacuum pump 32 shown in FIG. 1 and another end of the treatment object 21. Depending on materials, geometry and size of the treatment object 21, intake adapter 24 and the exhaust adapter 28 can be designed and manufactured, by changing appropriately the well-known gas joints or vacuum components.

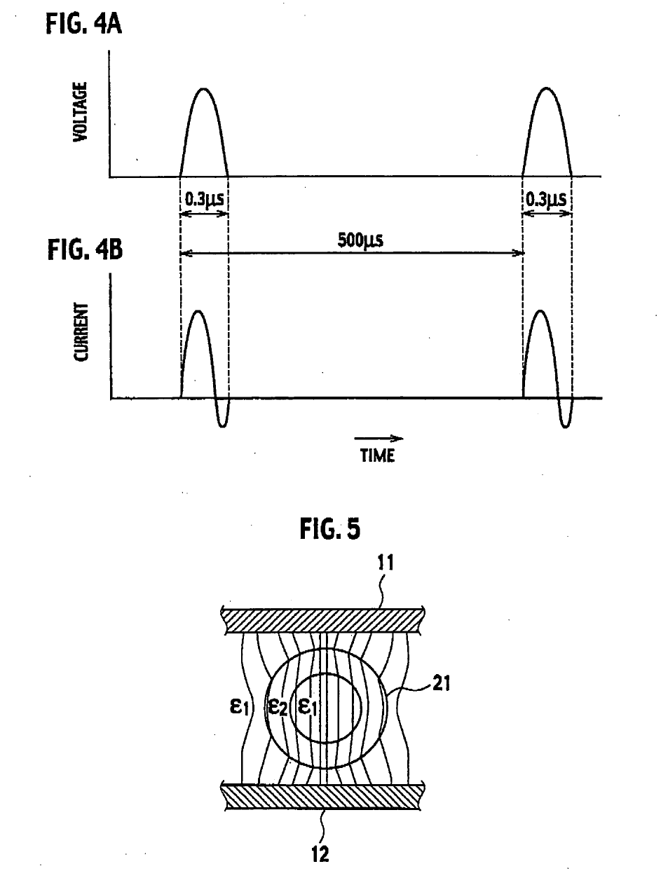

A high voltage pulse at high repetition rate as shown in FIG. 4A is applied across the first main electrode 11 and the second main electrode 12. FIG. 4A shows a pulse width of the voltage pulse measured at full width at half maximum. (FWHM) is 300 nano seconds, however for the pulse width of the main pulse, around 50-300 nano seconds is preferable. When, in the surface treatment apparatus related to the first embodiment, if a distance between the first main electrode 11 and the second main electrodes 12, implementing a parallel plate electrode, is 15 millimeters, a high voltage pulse with a repetition frequency of 2 k Hz, and voltage value of around 24 kV is preferred. In addition, as for the pressure in the treatment object 21, about 30 kPa, and the nitrogen gas flow rate, around 1 SLM is preferred. Because the repetition period is 500 microseconds as shown in FIG. 4, and, in the case of the repetition frequency 2 k Hz for the high voltage pulse, the duty ratio becomes 0.3/500=0.006. Therefore, non-thermal equilibrium low temperature plasma is generated efficiently and stably, without generating heat plasma as the high frequency discharge generates.

In the surface treatment apparatus related to the first embodiment of the present invention, duty ratio of 10⁻⁷ to 10⁻¹ is preferable for the voltage pulse. If the duty ratio is less than 10⁻⁷, the discharge becomes unstable, and if the duty ratio is more than 10⁻¹, unfavorable effect of heat plasma becomes prominent. The duty ratio of around 0.003-0.01 is more preferable. In addition, even a barrier discharge by a low frequency alternating electric field can be used to generate low temperature plasma in the treatment object 21, but a large input power cannot be expected by the barrier discharge.

Even for finely machined optical system or medical instrument such as an endoscope, which includes metallic components, because the duty ratio can be set to be around 10⁻⁷ to 10⁻¹ according to the surface treatment apparatus related to the first embodiment, metallic components will not rise to a considerable high temperature, and the optical system can overcome the problem that warp or misalignment is generated by thermal effect of the plasma.

When a treatment object 21 made of dielectric material is inserted between the first main electrode 11 arid the second main electrode 12, implementing a parallel plate electrode, and if dielectric constant ε₂ of the dielectric material is larger than dielectric ε₁ of gas (relative dielectric constant=1), the approximate electric field distribution can be represented as shown in FIG. 5. As for the electric field strength around the centerline extending vertically along the the center of the first main electrode 11 and the second main electrode 12 of FIG. 5, as illustrated by approximately parallel straight lines in FIG. 5, the electric field in the inside of the treatment object 21 made of dielectric material becomes the same of the electric field in the outside of the treatment object 21.

Because the dielectric breakdown field depends upon the size of space, or if the ambient pressure at inside and outside of the treatment object 21 is the same, the dielectric breakdown field becomes large in the inside of the treatment object 21. Therefore, it is necessary to reduce the dielectric breakdown field in the treatment object 21, by an appropriate method, to generate discharge in the inside of the treatment object 21. One method is to reduce gas pressure in the inside of the treatment object 21, for discharge in the right side region of Paschen's curve.

### Second Embodiment

As shown in FIG. 6, a surface treatment apparatus related to a second embodiment of the present invention encompasses a process chamber (23, 53, 54,62) establishing a closed space enclosing the surrounding of the treatment object 21; and an ambient gas adjusting means (62,65, 66b, 25b), having the first main electrode 11b as the anode and the second main electrode 12 as the cathode, for supplying the process gas in the process chamber (23, 53, 54,62), from the first main electrode 11b like a shower toward the second main electrode 12, and evacuating the shower of the process gas from a part of the process chamber (23, 53, 54,62). The main pulse of duty ratio of 10⁻⁷ to 10⁻¹ is applied between the first main electrode 11b and second main electrode 12, and an outer surface of the treatment object 21 is treated in non-thermal equilibrium plasma.

In the surface treatment apparatus related to the second embodiment, the treatment object 21 has a tubular geometry made of dielectric material as shown in FIG. 6. A pulse power supply 14 applies electric pulses (main pulses) across the first main electrode 11b and the second main electrode 12, which implement a quasi-parallel plate electrode, so that the electric pulse can cause the fine-streamer discharge in the sealed up space, which surrounds the outside of the treatment object 21.

Because a periodic array of T-shaped protrusions, rather than flat slab configuration, is employed for the first main electrode 11b, we will call the electrode configuration shown in FIG. 6 as “quasi-parallel plate electrode” in view of the situation such that each of discharge points originates at each tips of the T-shaped protrusions, and all of the tips of the T-shaped protrusion are arranged on a single plane so as to implement a virtual flat slab. In this case the first main electrode 11b is equivalent to an array of bar-shaped (linear) electrodes arranged in parallel so as to implement a ladder, and the ladder can implement an approximately “parallel plate electrode” with the second main electrode 12.

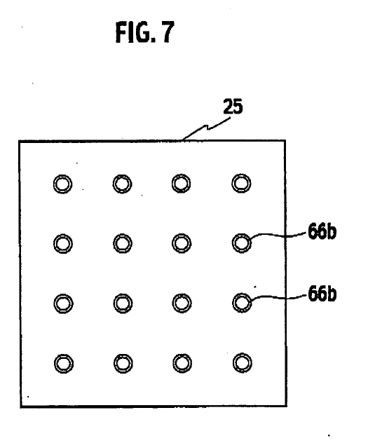

In addition, as the allocations of the exhausting piping 63 to be connected to the process chamber (23, 53, 54, 62), any site of the process chamber, rather than the down-stream side of the treatment object 21 shown in FIG. 6 can be employed. As shown in FIG. 6, the ambient gas adjustment mechanism (62, 65, 66b, 25b) embraces an injection-adjusting chamber 62, a gas supply layer 65 connected to injection-adjusting chamber 62, the first electrode protection layer (first main electrode protection layer) 25b. The gas supply layer 65 has a plurality of gas supply holes 66b arranged in a matrix form as shown in FIG. 7. The gas supply layer 65, which is made of porous ceramics, makes the flow of the treatment gas uniform. Six planes, which establish a flat rectangular parallelepiped, implement injection-adjusting chamber 62, the five planes but of six planes are made of metallic material, and the remaining one plane (in a cross-sectional view shown in FIG. 6, the left side plane) is substituted by the gas supply layer 65.

The ambient gas adjustment mechanism (62, 65, 66b, 25b) is implemented by a plurality of taper-shaped gas supply holes 66b penetrating through the first electrode protection layer (first main electrode protection layer) 25b, as shown in FIG. 7, the gas supply holes 66b are arranged in a form of two-dimensional matrix with a redetermined pitch. On the other hand, on the second electrode (second main electrode) 12, a second electrode covering insulator (second main electrode covering insulator) 23 of high purity quartz glasses is disposed.

The process chamber (23, 53, 54, 62), so as to implement four planes of a rectangular parallelepiped, embraces the second electrode covering insulator (second main electrode covering insulator) 23, a chamber top lid 53, a chamber bottom lid 54 and an injection-adjusting chamber 62, and two side plates at a rearward portion of the paper (not illustrated) and at the near side (not illustrated) of the paper of FIG. 6, implement remaining two planes of the rectangular parallelepiped. To the chamber bottom lid 54 and the chamber top lid 53, a top treatment object holder 52 and a bottom treatment object bolder 51 are attached, respectively, so as to implement a sealed up space. To establish, the sealed up space, the top treatment object holder 52 holds one end (upper-stream side) of the treatment object 21, which is connected to the chamber top lid 53, the bottom treatment object holder 51 holds another end (down-stream side) of the treatment object 21, which is connected to the chamber bottom lid 54. Depending on materials, geometry and size of the treatment object 21, by applying required changes and modifications appropriately, the structure of the top treatment object holder 52 and the bottom treatment object holder 51 can be designed and manufactured with well-known gas joint or vacuum components, easily.

Furthermore, as shown in FIG. 6, the surface treatment apparatus related to the second embodiment embraces a gas source 33 such as gas cylinders configured to store process gas, an injecting piping 61 connected to the gas source 33, and an injecting valve 41 connected to the injecting piping 61. In addition, though the Although the illustration is omitted, to at least one of the top treatment object holder 52 and tile bottom treatment object holder 51, the valve for gas introduction may be provided.

In the process chamber (23, 53, 54, 62), through the injecting piping 61, injecting valve 41 and the injection-adjusting chamber 62, process gas is supplied from gas source 33, and the flow of the process gas is shaped configuration of uniform shower by the ambient gas adjustment mechanism (62, 65, 66b, 25b). The process gas supplied to inside of the process chamber (23, 53, 54, 62) from the ambient gas adjustment mechanism (62, 65, 66b, 25b) is exhausted by the exhausting piping 63 from the process chamber (23, 53, 54, 62).

Therefore, as shown in FIG. 6, a vacuum pump 31 configured to evacuate the process chamber (23, 53, 54, 62) through the exhausting piping 63 connected to the process chamber at another end (down-stream side) side of the tubular treatment object 21 is provided to the surface treatment apparatus related to the second embodiment. The vacuum pump 31, through the exhausting piping 63 and the exhausting valve 42, is connected to the process chamber (23, 53, 54, 62). It is preferable, for the exhausting valve 42, to use the variable conductance valve through which the exhaust conductance can be adjusted.

In FIG. 6, the case that the second main electrode 12 is grounded so as to function as the cathode, while high voltage is applied to the first main electrode 11b so as to function as an anode is illustrated. Polarity of the pulse power supply 14 can be reversed, such that the first roam electrode 11b is assigned as the cathode, and the second main electrode 12 is assigned as the anode. When the first main electrode 11b is assigned as the cathode, the first main electrode 11b is grounded as a slab-shaped electrode, and a high voltage is applied to the second main electrode 12, and the ambient gas adjustment mechanism (62, 65, 66b, 25b) is provided to the second main electrode 12.

Similar to the first embodiment, a narrow tube having an inside diameter of less than or equal to 7-5 millimeters and a length of more than 4-7 meters may serve as the tubular treatment object 21 in the surface treatment apparatus related to the second embodiment. However, if the length is equal to or less than 4 meters, and inside diameter is more than 7 millimeters, the tube can be similarly processed. In addition, a cross-section of the treatment object 21 is not limited to a circular geometry, as already explained in the first embodiment.

Although the illustration is omitted, if the treatment object 21 is a flexible long-narrow tube, by providing first and second reels which roll up the treatment object 21, one end of the treatment object 21 may be rewound from the first reel so that another end of the treatment object 21 can be rolled up by the second reel, and surface treatment of the outside of the treatment object 21 may be executed partially and sequentially.

In the surface treatment apparatus related to the second embodiment, a high purity nitrogen gas could be supplied as the process gas through the ambient gas adjustment mechanism (62, 65, 66b, 25b) in a shape of a shower, however the “process gas” is not always limited to nitrogen gas. For example, for pasteurize or sterilize the outer surface of the treatment object 21, mixed gas of nitrogen gas with various kinds of active gas, which may include halogen based compound gas, can be adopted.

A high voltage pulses with high repetition rate as shown in FIG. 4A is applied across the first main electrode 11b and the second main electrode 12. FIG. 4A shows an example of pulse width spanning in a range of 10-500 nano seconds, which is preferable for the main pulse. When, in the surface treatment apparatus related to the second embodiment, if a distance between the first main electrode 11b and the second main electrodes, implementing a quasi-parallel plate electrode, is 15 millimeters, for the high voltage pulse with a repetition frequency of 2 kHz, a voltage value of around 24 kV is preferred.

Because a period is 500 microseconds, as shown in FIG. 4A, at a repetition frequency of 2 k Hz for the high voltage pulse, the duty ratio becomes 0.3/500=0.006, non-thermal equilibrium low temperature plasma is generated efficiently and stably, without generating heat plasma a ascribable to the high frequency discharge. A reasonable pulse width has a close relation to be close to distance between anode and cathode. From the start, along with the voltage application time, the discharge progress from the glow discharge to the streamer discharge, from the streamer discharge to the fine-streamer discharge, and from the fine-streamer discharge to the arc discharge. A discharge, which can maximize the plasma-input power, without reaching to the arc discharge, which is accompanied by high electric current, thermal dissipation and loss of electrode, is considered to be the fine-streamer discharge. Therefore, there is an appropriate pulse width to generate the fine-streamer discharge. It is ideal that the distance between anode and cathode, the discharged condition should be adjusted so that there is no application of voltage pulses, before reaching to the arc discharge.

To generate discharge in the sealed up space surrounding the outside of the treatment object 21, the injecting valve 41 and the exhausting valve 42 are adjusted so that internal gas pressure P2 of the process chamber (23, 53, 54, 62) is equal to the atmospheric pressure P3=101 kPa, or around 80-90 kPa, which is slightly lower than the atmospheric pressure P3. Under the condition such that, in the process chamber (23, 53, 54, 62), through the injecting piping 61 and the injecting valve 41, the process gas is supplied from the gas source 33, if high voltage pulses with high repetition rate as shown in FIGS. 4A and 4B are applied across the first main electrode 11b and the second main electrode 12, while the process gas is supplied as a shower by the ambient gas adjustment mechanism (62, 65, 66b, 25b), the non-thermal equilibrium low temperature plasma is generated in the inside of the process chamber (23, 53, 54, 62) by the fine-streamer discharge, the surface treatment of the outside of the treatment object 21 is achieved.

### First Modification of the Second Embodiment

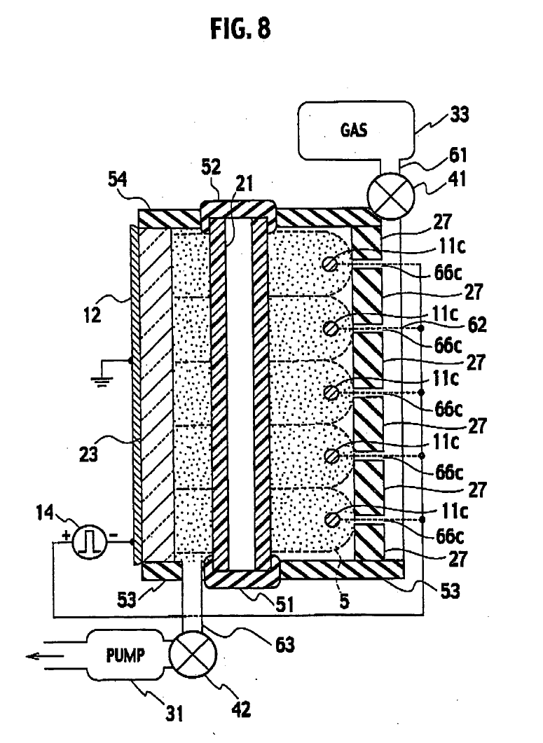

As shown in FIG. 8, a surface treatment apparatus related to a modification of the second embodiment of the present invention encompasses a process chamber (23, 53, 54,62) establishing a closed space enclosing the surrounding of the treatment object 21; and an ambient gas adjusting means (62, 27, 66c), having tile first main electrode 11c as the anode and the second main electrode 12 as the cathode, for supplying the process gas in the process chamber (23, 53, 64,62), from the first main electrode 11c like a shower toward the second main electrode 12, and evacuating the shower of the process gas from a part of the process chamber (23, 53, 54,62), The main pulse is applied between the first main electrode 11c and second main electrode 12, and the outside of the treatment object 21 is treated in non-thermal equilibrium plasma.

An array of first main electrodes 11c implement a periodic ladder structure, which arranges an array of bar (linear) electrodes, as shown in FIG. 8, can be regarded as “quasi-parallel plate electrode” with the second main electrode 12. Similar to the configuration shown in FIG. 6, the process chamber (23, 53, 54, 62), so as to implement four planes of a rectangular parallelepiped, embraces the second electrode covering insulator (second main electrode covering insulator) 23 and the process chamber bottom lid 53, the chamber bottom lid 54 and the injection-adjusting chamber 62, two side plates at a rearward portion of the paper (not illustrated) and at the near side (not illustrated) of the paper of FIG. 8, implement remaining two planes of the rectangular parallelepiped.

The second main electrode 12 serves as the cathode, and the surface treatment apparatus related to the first mortification of the second embodiment supplies the process gas as a shower from the first main electrode 11c serving as an anode, the structure of the ambient gas adjustment mechanism (62, 27, 66c) to exhaust the process gas from the exhausting piping 63 is different from the process chamber (23, 53, 54, 62) shown in FIG. 6.

The ambient gas adjustment mechanism (62, 27, 66c) embraces a process chamber side wall 27, to which a plurality of gas supply holes 66c are provided, and an injection-adjusting chamber 62, the process gas is injected from the injection-adjusting chamber 62 as shown in FIG. 8, which is exposed to discharge space a plurality of bar-shaped first main electrode 11c made of metal such as tungsten (W), parakeet flannel, it is in a problem pollution by metal, the if it is a problem and the use that are not done by pollution by metal, it is simpler and easy than structure shown in FIG. 6 and it is cheap and is advantageous in that it can be produced.

The plurality of gas simply holes 66c are arranged in two-dimensional matrix with uniform pitch, the gas supply holes 66c penetrate through the process chamber aide wall 27, as shown in FIG. 7. On the other hand, on the second electrode (second main electrode) 12, the second electrode covering insulator (second main electrode covering insulator) 23 of high purity quartz glasses is disposed.

Furthermore, the surface treatment apparatus related to the first modification of the second embodiment embraces a gas source 33 such as gas cylinders configured to store process gas, an injecting piping 61 connected to the gas source 33, an injecting valve 41 connected to the injecting piping 61 as shown in FIG. 8. It is preferable to adept a needle valve configured to adjust the flow rate for the injecting valve 41.

In the process chamber (23, 53, 54, 62), through the injecting piping 61 and the injecting valve 41, process gas is supplied from the gas source 33, and the flow of the process gas is shaped into the configuration of uniform shower by the ambient gas adjustment mechanism (62, 27, 66c). The process gas supplied by the ambient gas adjustment mechanism (62, 27, 66c) is exhausted by the exhausting piping 63 from the process chamber (23, 53, 54, 62). Then, as shown in FIG. 8, a vacuum pump 31 configured to evacuate inside of the process chamber (23, 53, 54, 62) at another end (down-stream side) side of the tubular treatment object 21 is provided to the surface treatment apparatus related to the first modification of the second embodiment.

The vacuum pump 31, through the exhausting piping 63 and the exhausting valve 42, is connected to the process chamber (23, 53, 54, 62). It is preferable for the exhausting valve 42 to use the variable conductance valve through which the exhaust conductance can be adjusted. To establish the sealed up space, the top treatment object holder 52 holds one end (upper-stream side) of the tubular treatment object 21 is connected to the chamber bottom lid 54, the bottom treatment object holder 51 holds another end (down-stream side) of the treatment object 21, which is connected to the chamber bottom lid 54. Depending on materials, geometry and size of the treatment object 21, by applying required changes and modifications appropriately, the structure of the top treatment object holder 52 and the bottom treatment object holder 51 can be designed and manufactured with well-known gas joint or vacuum components, easily.

In FIG. 8, the second main electrode 12 is grounded so as to serve as the cathode, while a high voltage is applied to the first main electrode 11c, which is used as an anode is illustrated, however the polarity of pulse power supply 14 is reversed so that the first main electrode 11c serve as the cathode, and the second main electrode 12 serve as anode. When the first main electrode 11c is assigned as the cathode, which is grounded, the first main electrode 11c may be implemented by a slab-shaped electrode, a high voltage is applied to the second main electrode 12, and the ambient gas adjustment mechanism (62, 27, 66c) embraces the second main electrode 12.

Similar to the first embodiment, a narrow tube having an inside diameter of less than or equal to 7-5 millimeters and a length of more than 4-7 meters may serve as the tubular treatment object 21 in the surface treatment apparatus related to the first modification of the second embodiment. However, even if the length is less than 4 meters, and inside diameter is more than 7 millimeters inside diameter, the treatment object 21 can be processed. In addition, a cross-section of the treatment object 21 is not limited to a circular geometry, as already explained in the first embodiment.

Although the illustration is omitted, if the treatment object 21 is a flexible long-narrow tube, by providing first and second reels which roll up the treatment object 21, the treatment object 21 may be rewound from the first reel so that the treatment object 21 can be rolled up by the second reel, and surface treatment of the outside of the treatment object 21 may be executed partially and sequentially.

In the surface treatment apparatus related to the first modification of the second embodiment, a high purity nitrogen gas can be supplied as the process gas through the ambient gas adjustment mechanism (62, 27, 66c), however the “process gas” is not always limited to nitrogen gas. For example, for pasteurization or sterilization, mixed gas of nitrogen gas with various kinds of active gas such as halogen based compound gas can be adopted.

A high voltage pulses with high repetition rate as shown in FIGS. 4A and 4B is applied to between the first main electrode 11c and the second main electrode 12. FIG. 4A shows a pulse having the pulse width around 10-500 nanoseconds, which is preferable for the main pulse. When, in the surface treatment apparatus related to the first modification of the second embodiment, distance of between the first main electrode 11c and the second main electrode 12, which implements a quasi-parallel plate electrode, is 15 millimeters, as the high voltage pulse with a repetition frequency of 2 kHz, a voltage value of around 24 kV is preferred.

Because a period is 500 microseconds, as shown in FIGS. 4A and 4B, at a repetition frequency of 2 k Hz for the high voltage pulse, the duty ratio becomes 0.8/500=0.006, non-thermal equilibrium low temperature plasma is generated efficiently and stably, without generating heat plasma ascribable to the high frequency discharge.

To generate discharge in the sealed up space surrounding the outside of the treatment object 21, the injecting valve 41 and the exhausting valve 42 are adjusted so that internal gas pressure P2 of the process chamber (23, 53, 54, 62) is equal to the atmospheric pressure P3=101 kPa, or around 80-90 kPa, which is slightly lower than the atmospheric pressure P3. Under the condition such that, in the process chamber (23, 53, 54, 62), through the injecting piping 61 and the injecting valve 41, the process gas is supplied from the gas source 33, if high voltage pulses with high repetition rate as shown in FIGS. 4A and 4B are applied across the first main electrode 11b and the second main electrode 12, while the process gas is supplied as a shower by the ambient gas adjustment mechanism (62, 27, 66c), the non-thermal equilibrium low temperature plasma is generated in the inside of the process chamber (23, 53, 54, 62) by the fine-streamer discharge, the surface treatment of the outside of the treatment object 21 is achieved.

### Second Modification of the Second Embodiment

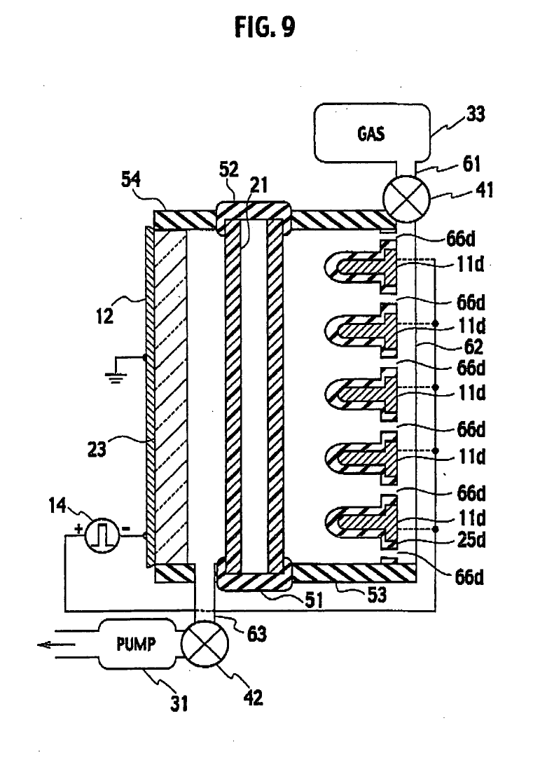

As shown in FIG. 9, a surface treatment apparatus related to a modification of the second embodiment of the present invention encompasses a process chamber (23, 53, 54,62) establishing a closed space enclosing the surrounding of the treatment object 21; and an ambient gas adjusting means (62, 25d, 66d), having the first main electrode 11c as the anode and the second main electrode 12 as the cathode, for supplying the process gas in the process chamber (23, 53, 54,62), from the first main electrode 11c like a shower toward the second main electrode 12, and evacuating the shower of the process gas from a part of the process chamber (23, 53, 54,62). The main pulse is applied between the first main electrode 11c and second main electrode 12, and the outside of the treatment object 21 is treated in non-thermal equilibrium plasma.

A plurality of T-shaped protrusions rather than flat slab configuration the first main electrode 11d is arranged as shown in FIG. 9 so as to implement the “quasi-parallel plate electrode”, in view of the situation such that each of discharge points originates at each tips of the T-shaped protrusions. The second main electrode 12 serves as the cathode, and the surface treatment apparatus related to the second modification of the second embodiment supplies the process gas as a shower from the first main electrode 11d side as an anode, the structure of the ambient gas adjustment mechanism (62, 25d, 66d) to exhaust the process gas from the exhausting piping 63 from the process chamber (23, 53, 54, 62) is different from structure shown in FIG. 6.

In the first modification shown in FIG. 8, pollution by metal became a problem because the first main electrode 11c made of metal such as tungsten (W), is exposed in the discharge space, in the surface treatment apparatus related to the second modification of the second embodiment of the present invention, the first electrode protection layer (first main electrode protection layer) 25d of alumina covers the surface of the first main electrode 11c, and the pollution by metal is controlled. The ambient gas adjustment mechanism (62, 25d, 66d) embraces a plurality of gas supply holes 66d established in the injection-adjusting chamber 62, the first electrode protection layer (first main electrode protection layer) 25d, as shown in FIG. 9. A plurality of gas supply holes 66d are arranged in a configuration similar to the layout shown in FIG. 7, that is, they are arranged in a form of two-dimensional matrix with uniform pitch. On the second electrode (second main electrode) 12, the second electrode covering insulator (second main electrode covering insulator) 23 of high purity quartz glasses is disposed.

Since other functions, configurations, and way of operation are substantially similar to the functions, configurations, and way of operation already explained in the second embodiment with FIG. 6, overlapping or redundant description may be omitted.

### Third Embodiment

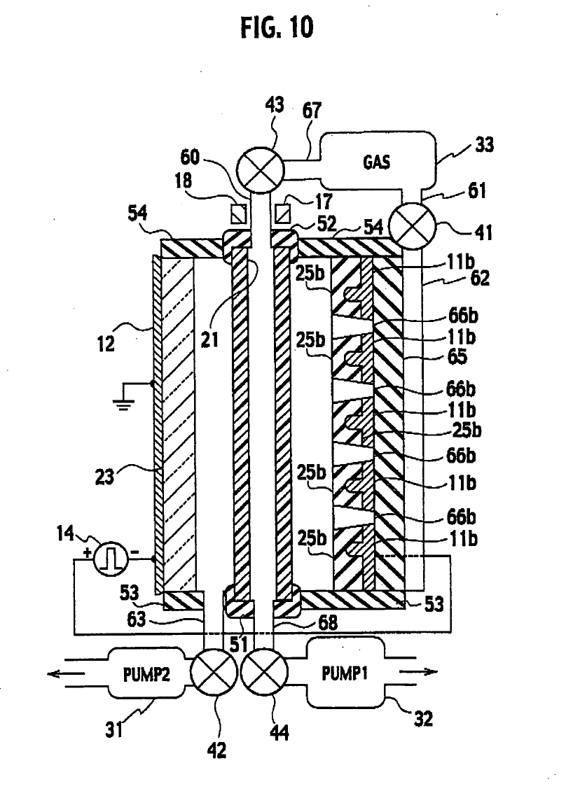

A plurality of T-shaped protrusions, rather than flat slab configuration, are arranged so as to implement the “quasi-parallel plate electrode” as shown in FIG. 10, for the first main electrode 11b. Each of the discharge points originates at each tips of the T-shaped protrusions. In this case, as for the first main electrode 11b, the periodical ladder shaped electrode which is implemented by a plurality of bar (linear) electrodes, has been explained in the second embodiment, as a whole, the structure implemented by the first main electrode 11b and the second main electrode 12 is approximately “parallel plate electrode”.

The surface treatment apparatus related to the third embodiment goes to the second main electrode 12 side as the cathode in the process gas from the first main electrode 11b side as an anode Similar to the surface treatment apparatus related to the second embodiment, and it supplies in the shape of a shower, further encompasses the ambient gas adjustment mechanism (62, 65, 66b, 25b) to exhaust the process gas from the second exhausting piping 63 from the process chamber (23, 63, 54, 62), it is different from the surface treatment apparatus related to the first embodiment.

The process chamber (23, 53, 54, 62), so as to implement four planes of a rectangular parallelepiped, embraces a second electrode covering insulator (second main electrode covering insulator) 23, a chamber top lid 53, a chamber bottom lid 54 and an injection-adjusting chamber 62, two side plates at a rearward portion of the paper (not illustrated) and at the near side (not illustrated) of the paper of FIG. 10, implement remaining two planes of the rectangular parallelepiped.

There is no by the rectangular parallelepiped which is flatness, and the injection-adjusting chamber 62 embraces metallic five plane out of six planes of a rectangular parallelepiped, the gas supply layer 65 substitutes one plane (a cross-sectional view shown in FIG. 10, the left side plane). The ambient gas adjustment mechanism (62, 65, 66b, 25b) embraces an the injection-adjusting chamber 62, a gas supply layer 65 made of porous ceramics making the process gas from the injection-adjusting chamber 62 is distributed uniformly, a gas supply layer 65 as shown in FIG. 10, and a first electrode protection layer (first main electrode protection layer) 25b having a plurality of gas supply holes 66b. The ambient gas adjustment mechanism (62, 65, 66b, 25b) is implemented by a plurality of taper-shaped gas supply holes 66b penetrating through the first electrode protection layer (first main electrode protection layer) 25b, as shown in FIG. 7, the gas supply holes 66b are arranged in a form of two-dimensional matrix with a predetermined pitch. On the other hand, on the second electrode (second main electrode) 12, the second electrode covering insulator (second main electrode covering insulator) 23 of high purity quartz glasses is disposed.

Furthermore, the surface treatment apparatus related to the third embodiment embraces a gas source 33 such as gas cylinders configured to store process gas, a first injecting piping 67 connected to the gas source 33, a second injecting piping 61 connected to the gas source 33, a first injecting valve 43 connected to second injecting piping 67, and a second the injecting valve 41 connected to the second injecting piping 61 as shown in FIG. 10. It is preferable to adopt a needle valve configured to adjust the flow rate for the first injecting valve 43, the second injecting valve 41.

The first injecting piping 67 and the first injecting valve 43, process gas is supplied from the gas source 33 in the inside of the tubular treatment object 21, and the process gas is supplied by the upper stream side, by vacuum pump (second pump) 31 that comprised downstream, the process gas drifts to the treatment object 21, the treatment object 21 is near in an the atmospheric pressure of around 20-30 kPa, the pressure is kept at a processing pressure of less than or equal to an the atmospheric pressure. On the other hand, in the process chamber (23, 53, 54, 62), the second injecting piping 61 and the second injecting valve 41, process gas is supplied from the gas source 33, and the flow of the process gas is shaped into the configuration of uniform shower by the ambient gas adjustment mechanism (62, 65, 66b, 25b).

The process gas supplied by the ambient gas adjustment mechanism (62, 65, 66b, 25b) is exhausted by the second exhausting piping 63 from the process chamber (23, 53, 54, 62). Then, as shown in FIG. 10, the second vacuum pump (second pump) 31 configured to evacuate space surrounding the outside of the treatment object 21 in another end (down-stream side) side of the tubular treatment object 21 is provided to the surface treatment apparatus related to the third embodiment. The second vacuum pump (second pump) 31 is connected to the second exhausting piping 63 and the second exhausting valve 42, is connected to the process chamber (23, 53, 54, 62). On the other hand, the first vacuum pump (first pump) 32 is connected to exhausting piping 68 and the first exhausting valve 44, is connected to another end (down-stream side) of the treatment object 21. It is preferable for the first exhausting valve 44 and the second exhausting valve 42 to use the variable conductance valve through which the exhaust conductance can be adjusted.

To establish, the sealed up space, the top treatment object holder 52 holds one end (upper-stream side) of the tubular treatment object 21 is connected to the chamber bottom lid 54, the bottom treatment object holder 51 holds another end (down-stream side) of the treatment object 21, which is connected to the chamber bottom lid 54. Depending on materials, geometry and size of the treatment object 21, by allying required changes and modifications appropriately, the structure of the top treatment object holder 52 and the bottom treatment object holder 51 can be designed and manufactured with well-known gas joint or vacuum components, easily.

In FIG. 10, the second main electrode 12 is grounded so as to serve as the cathode, the case that high voltage is applied to the first main electrode 11b, and was used as an anode is illustrated, it turns over by polarity of pulse power supply 14 and is preferable in anode, the first main electrode 11b in the second main electrode 12 as the cathode. When the first main electrode 11b is assigned as the cathode, the first main electrode 11b is made into a slab-shaped electrode, and is grounded, the high voltage is applied as a type electrode to enjoy at a couple of the second main electrode 12, and the ambient gas adjustment mechanism (62, 65, 66b, 25b) is provided to the second main electrode 12.

Similar to the first embodiment, a narrow tube having an inside diameter of less than or equal to 7-5 millimeters and a length of more than 4-7 meters may serve as the tubular treatment object 21 in the surface treatment apparatus related to the third embodiment as well, even if the length is equal to or less than 4 meters, and inside diameter is more than 7 millimeters, the tube can be similarly processed. In addition, a cross-section of the treatment object 21 is not limited to a circular geometry, as already explained in the first embodiment.

Although the illustration is omitted, if the treatment object 21 is a flexible long-narrow tube, by providing first and second reels which roll up the treatment object 21, the treatment object 21 may be rewound from the first reel so that the treatment object 21 can be rolled up by the second reel, and internal surface treatment of the treatment object 21 may be executed partially and sequentially.

In FIG. 10, the excited particle supplying system (17,18) encompasses a first auxiliary electrode 17 and a second auxiliary electrode 18, which sandwich the feed piping 60 connected to the upper-stream side of the treatment object 21, implementing a parallel plate electrode, an auxiliary pulse power supply (although the illustration is omitted) configured to apply a voltage pulse (a supporting pulse) across the first auxiliary electrode 17 and the second auxiliary electrode 18 so as to generate initial plasma, as already explained in the first embodiment The feed piping 60 is a piping made of dielectric material.

If initial plasma can be supplied after the flow of gas in the early stage an excited particle supplying system discharges electricity, and to start, the what may activate initial plasma by inductive plasma source rather than a thing limited to the parallel plate electrode configuration that seems to have always illustrated in FIG. 10 structure of others is similar for the case the surface treatment apparatus related to the first embodiment. After excitation of initial plasma, the surface treatment apparatus shown in FIG. 10 processes inside of the tubular treatment object 21 and top surface by radicals included in plasma.

In the surface treatment apparatus related to the third embodiment, a high purity nitrogen gas can be supplied as the process gas in the treatment object 21 from the upper-stream side, the “the process gas” is not always limited to nitrogen gas. For example, for inside of the treatment object 21 and objects such as pasteurization or sterilization, mixed gas of nitrogen gas with various kinds of active gas such as halogen based compound gas can be adopted.

A high voltage pulses with high repetition rate as shown in FIGS. 4A and 4B is applied across the first main electrode 11 and the second main electrode 12. FIG. 4A shows pulse width of 10-500 nano seconds preferable for the main pulse. When, in the surface treatment apparatus related to the third embodiment, if a distance between the first main electrode 11b and the second main electrodes, implementing a quasi-parallel plate electrode, is 15 millimeters, for the high voltage pulse with a repetition frequency of 2 kHz, a voltage value of around 24 kV is preferred.

Because a period is 500 microseconds, as shown in FIGS. 4A and 4B, at a repetition frequency of 2 k Hz for the high voltage pulse, the duty ratio becomes 0.3/500=0.006, non-thermal equilibrium low temperature plasma is generated efficiently and stably, without generating heat plasma ascribable to the high frequency discharge.

### <Three Operation Modes>

In the surface treatment apparatus related to the third embodiment, there are three operation modes. That is to say, a first mode configured to ignite an discharge only in the inside of the treatment object 21, a second mode configured to ignite an discharge only at the outside of the treatment object 21, and a third mode configured to ignite an discharge both inside and outside of the treatment object 21 having tubular geometry.

### (a) First Mode:

When it was parallel, and, as described in the surface treatment apparatus related to the first embodiment, the treatment object 21 made of dielectric material was put in an electrode side in the first main electrode 11b and the second main electrode 12 implementing a parallel plate electrode, the if dielectric constant ε2 of a dielectric is larger than dielectric constant ε1 of gas, the, as for the electric field distribution of an approximately, an dielectric breakdown field becomes large in the treatment object 21 like FIG. 5.

Therefore, The internal gas pressure P1 in the treatment object 21 is made around 10-40 kPa in the inside of the treatment object 21 made of dielectric material in order to be caused, and it is desirable to lower than outside gas pressure P2 of the treatment object 21.

And it is more extremely than the atmospheric pressure P3 slightly desirable for around 80-90 kPa to lower whether outside gas pressure P2 of the treatment object 21 is equal with the atmospheric pressure P3=101 kPa. If the first injecting valve 43, the second injecting valve 41, the first exhausting valve 44 and the second exhausting valve 42 are that is to say adjusted for the purpose of becoming:

P1<P2≦P3 (1).

Or gas pressure P1 of the treatment object 21 inside is turned into around 10-40 kPa and, the outside gas pressure P2 of the treatment object 21, the as pressure of less than or equal to 10⁻³ Pa to 10⁻⁵ Pa: The first injecting valve 43, the second injecting valve 41, the first exhausting valve 44 and the second exhausting valve 42 may be adjusted for the purpose of becoming:

P2<<P1<P3 (2).

Because of this, for example, the first pressure gauge is provided to exhausting piping 68 and the second exhausting piping 63, the first injecting valve 43, the second injecting valve 41, the first exhausting valve 44 and the second exhausting valve 42 may be adjusted by return control. Or the first injecting piping 67 and a mass-flow controller controlling flow rate in the second injecting piping 61 may be arranged. The first pressure gauge may be provided to injecting valve 43 and each down stream side of the second injecting valve 41. After having set a pressure condition as shown in an in an Eq. (1) or (2), the second injecting valve 41 and the second exhausting valve 42 are closed, the outside gas-flow of the treatment object 21 is left, the gas-flow is formed only in the inside of the treatment object 21.

And an excited particle supplying system (17,18) is started, and initial plasma is supplied after the flow of gas, the if high voltage pulses with high repetition rate as shown in FIGS. 4A and 4B is applied across the first main electrode 11 and the second main electrode 12 more, the, non-thermal equilibrium low temperature plasma style, is transported inside of the treatment object 21, the surface treatment of inside of the treatment object 21 is achieved.

### (b) Second Mode:

Gas pressure P1 of the treatment object 21 inside is set in a value of extra cost of comparison of around 70-90 kPa only at the outside of the treatment object 21 in order to be caused, the few is more extremely than outside gas pressure P2 of the treatment object 21 low or, it will be done in approximately the same degree. And if outside gas pressure P2 of the treatment object 21 is more extremely than the atmospheric pressure P3 slightly lowered to around 80-90 kPa with the atmospheric pressure PS=101 kPa whether it is equal. If the first injecting valve 43, the second injecting valve 41, the first exhausting valve 44 and the second exhausting valve 42 are that is to say adjusted for the purpose of becoming:

P1≦P2≦P3 (3).

But it is not necessary to be low, and gas pressure P1 of the treatment object 21 inside always does gas pressure P1 of the treatment object 21 inside larger than the atmospheric pressure P3 with the atmospheric pressure P3=101 kPa than outside gas pressure P2 of the treatment object 21 whether you are approximately equal, the outside gas pressure P2 of the treatment object 21 is equal with the atmospheric pressure P3, too as well or, the few seems to be more extremely than the atmospheric pressure P3 lowered to around 80-90 kPa: It is preferable as,

P2≦P1→P3 (4)

P2≦P3<P1 (5).

Or gas pressure P1 of the treatment object 21 inside is turned into pressure of less than or equal to 10⁻³ Pa to 10⁻⁵ Pa and is equal with the atmospheric pressure P3=101 kPa in outside gas pressure P2 of the treatment object 21 or, the as pressure of around 80-90 kPa: The first injecting valve 43, the second injecting valve 41, the first exhausting valve 44 and the second exhausting valve 42 may be adjusted for the purpose of becoming:

P1<<P2≦P3 (6).

After having set pressure conditions as shown by Eqs. (3)-(6), the first injecting valve 43 and the first exhausting valve 44 are closed, internal gas-flow of the treatment object 21 is left. And if, in the process chamber (23, 53, 54, 62), the second injecting piping 61 and the second injecting valve 41, process gas is supplied from the gas source 33, and the process gas applies high voltage pulses with high repetition rate as shown in FIGS. 4A and 4B in between the first main electrode 11 and the second main electrode 12 in a state supplied m the shape of a shower from the ambient gas adjustment mechanism (62, 65, 66b, 25b), the non-thermal equilibrium low temperature plasma is generated in the outside of the treatment object 21 by the fine-streamer discharge, the surface treatment of the outside of the treatment object 21 is achieved. With a mode making an discharge cause only at the outside of the treatment object 21, excited particle supplying system (17,18) will not started, of course.

### (c) Third Mode:

The treatment object 21 internal gas pressure P1 is made around 10-40 kPa in order to make an discharge cause in the inside and outside both the treatment object 21, audit is desirable to lower than outside gas pressure P2 of the treatment object 21. And outside gas pressure P2 of the treatment object 21 is more extremely than the atmospheric pressure P3 slightly lowered to around 80-90 kPa with the atmospheric pressure P3=101 kPa whether it is equal, and it seems to be in a pressure condition as shown by Eq. (1), the first injecting valve 43, the second injecting valve 41, the first exhausting valve 44 and the second exhausting valve 42 are adjusted.

After having set in a pressure condition as shown by Eq. (1), the excited particle supplying system (17,18) is started, and initial plasma is supplied after the flow of gas, the if high voltage pulses with high repetition rate as shown in FIGS. 4A and 4B applies to between the first main electrode 11 and the second main electrode 12 more, the, non-thermal equilibrium low temperature plasma style, is transported inside of the treatment object 21, the at the same time as surface treatment of inside of the treatment object 21 is achieved, in the process chamber (28, 58, 54, 62), the process gas is supplied in the shape of a shower by the ambient gas adjustment mechanism (62, 65, 66b, 25b), the fine-streamer discharge produces, the non-thermal equilibrium low temperature plasma, the of the treatment object 21, the is formed outside, the surface treatment of the outside of the treatment object 21 is achieved simultaneously, too.

### Fourth Embodiment

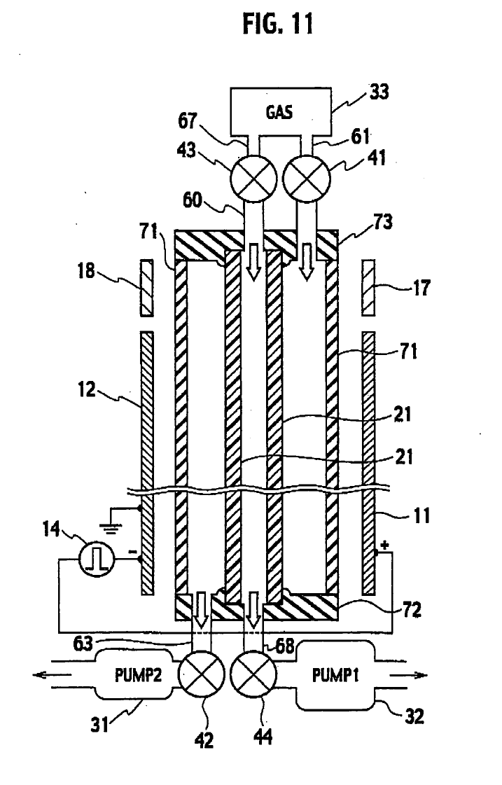

A surface treatment apparatus related to a fourth embodiment of the present invention prepares for accommodation tube 71 to receive the treatment object 21 of the tubular geometry that is a long-narrow tube as shown in FIG. 11, the plasma style is drained into inside of the treatment object 21 and outside both, inside and the outside of the treatment object 21 may be processed simultaneously.

The surface treatment apparatus related to other embodiment that is to say embraces a second the injecting valve 41 that the first injecting valve 43 and the second injecting piping that the first injecting piping is provided, and is connected to gas source 33 and a gas source 33 such as gas cylinders configured to store process gas, process gas is supplied, and is connected as shown in FIG. 11. The first injecting valve 43 is provided from the source 33 in the inside of the tubular treatment object 21, and the process gas is supplied by upper-stream side, by vacuum pump (first pump) 32 that comprised downstream, the process gas drifts to the treatment object 21, the treatment object 21 is near in an the atmospheric pressure of around 20-30 kPa, the pressure is kept at a processing pressure of less than or equal to an the atmospheric pressure.

On the other hand, in the process chamber (23, 53, 54, 62) encompasses accommodation tube 71 the second injecting valve 41 is provided from the gas source 33, and the process gas is supplied by the upper-stream side, by vacuum pump (second pump) 31 that comprised downstream, the process gas drifts to accommodation tube 71, the accommodation tube 71 is near in an the atmospheric pressure of around 80-90 kPa, the pressure is kept at a processing pressure of less then or equal to an the atmospheric pressure.

An accommodation tube top cap 73 and a accommodation tube bottom cap 72 are connected to the upper end and a bottom end of each accommodation tube 71 so that a vacuum exhausts sealing up air space between the outside of accommodation tube 71 and the tubular treatment object 21, the sealed up space of double pipe structure is composed.

Furthermore, it is confronted each other to put in accommodation tube 71 that received the tubular treatment object 21, and is disposed, the first auxiliary electrode 17 the first main electrode 11b composing a parallel plate electrode and the upper-stream side of the second main electrode 12 and accommodation tube 71 are caught, and implementing a parallel plate electrode and the second auxiliary electrode 18 are comprised.

The internal gas pressure P1 in the treatment object 21 is made around 10-40 kPa in the inside of the treatment object 21 and outside both tubular geometry in order to be caused, and it is desirable to lower than gas pressure P2 between the accommodation tube 71 and the treatment object 21. And gas pressure P2 between the accommodation tube 71 and the treatment object 21 seems to be more extremely than the atmospheric pressure P3 slightly lowered to around 80-90 kPa with the atmospheric pressure P3=101 kPa whether it is equal, the if the first injecting valve 43, the second injecting valve 41, the first exhausting valve 44 and the second exhausting valve 42 are adjusted.

After having set in a predetermined pressure condition, the excited particle supplying system (17,18) is started, and, after the flow of gas of neither sealed up space between the outside of in the treatment object 21 and accommodation tube 71 and the treatment object 21, each initial plasma is supplied, the if high voltage pulses with high repetition rate as shown in FIGS. 4A and 4B is applied across the first main electrode 11 and the second main electrode 12 more, the, non-thermal equilibrium low temperature plasma style, is transported each at inside of the treatment object 21 and the outside, the surface treatment of inside of the treatment object 21 and the outside is achieved simultaneously.

### Fifth Embodiment

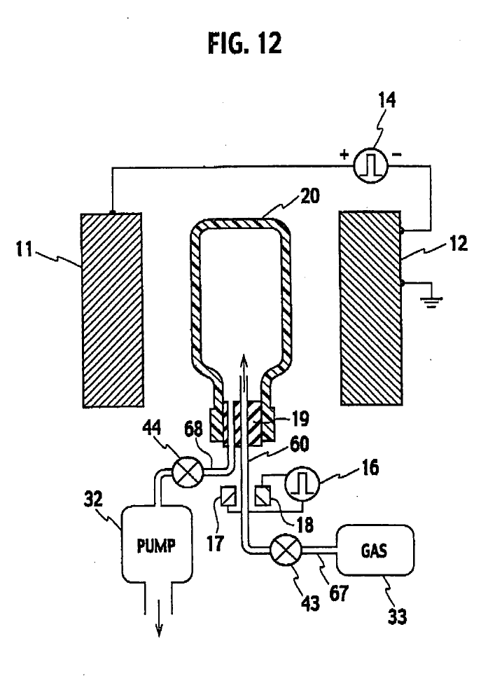

As shown in FIG. 12, a surface treatment apparatus related to a fifth embodiment of the present invention embraces a pot made of dielectric material, which serve as the treatment object 21, a neck adapter 19 inserted in the neck of the pot, a the feed piping 60 and an exhausting piping 68, which penetrate through the neck adapter 19. The feed piping 60 is a piping made of dielectric material.

The process gas is introduced in the inside of the pot-shaped treatment object 21 by the feed piping 60, the process gas is exhausted from the exhausting piping 68. The first main electrode 11 and the second main electrode 12, implementing a parallel plate electrode, facing each other so as to sandwich the treatment object 21.

In one part of the feed piping 60, an excited particle supplying system (16, 17 and 18) configured to supply initial plasma in the flow of gas for stating the discharge is provided. The excited particle supplying system (16, 17 and 18) embraces a first auxiliary electrode 17 and a second auxiliary electrode 18, implementing a parallel plate electrode, an auxiliary pulse power supply 16 configured to apply an electric pulse (a supporting pulse) across the first auxiliary electrode 17 and the second auxiliary electrode 18 so as to generate an initial plasma. On the other hand, the pulse power supply 14 applies an electric pulse (main pulse) across the first main electrode 11 and the second main electrode 12 to maintain the plasma in the inside of the treatment object 21, which is initiated by the initial plasma.

As shown in FIGS. 4A and 4B., a high voltage pulses with high repetition rate is applied. FIG. 4A shows pulse width of 10-500 nano seconds preferable for the main pulse. In FIG. 12, the second main electrode 12 is grounded so as to serve as the cathode, the case that high voltage is applied to the first main electrode 11, and was used as an anode is illustrated, it turns over by polarity of pulse power supply 14 and is preferable in anode, the first main electrode 11 in the second main electrode 12 as the cathode.

Furthermore, in the surface treatment apparatus related to the fifth embodiment, an injecting valve 43 is connected to the feed piping 60, an injecting piping 67 is connected to the injecting valve 43, a gas source 33 such as gas cylinders configured to store process gas is connected to the injecting piping 67. It is preferable to adopt a needle valve configured to adjust the flow rate for injecting valve 43. On the other hand, the process gas introduced by the feed piping 60 is exhausted vacuum pump 32. Therefore, an exhausting valve 44 is provided an exhausting piping 68, which is connected to the vacuum pump 32, so that the exhausting valve 44 can control the pressure at an appropriate processing pressure, when the flow of internal gas is introduced in the treatment object 21. It is preferable for the exhausting valve 44 to use the variable conductance valve through which the exhaust conductance can be adjusted.

The process gas is supplied from the gas source 33 in the inside of the pot-shaped treatment object 21 through the feed piping 60, which is inserted in the neck, such that the pressure is controlled at near the atmospheric pressure of around 20-30 kPa, or the pressure is controlled at a processing pressure of less than or equal to an the atmospheric pressure, in the treatment object 21, exhausting the process gas by vacuum pump 32 through the exhausting piping 68 that is inserted in the neck,

When, in the surface treatment apparatus related to the fifth embodiment, if a distance between the first main electrode 11 and the second main electrodes 12 implementing a parallel plate electrode, is 15 millimeters, for the high voltage pulse with a repetition frequency of 2 kHz, a voltage value of around 24 kV is preferred.

Because a period is 500 microseconds, as shown in FIGS. 4A and 4B, at a repetition frequency of 2 k Hz for the high voltage pulse, the duty ratio becomes 0.3/500=0.006, non-thermal equilibrium low temperature plasma is generated efficiently and stably, without generating heat plasma ascribable to the high frequency discharge.

In the surface treatment apparatus related to the fifth embodiment, a high purity nitrogen gas can be supplied as the process gas in the treatment object 21 from the neck, the “the process gas” is not always limited to nitrogen gas. For example, for pasteurize or sterilize inside of the treatment object 21, mixed gas of nitrogen gas with various kinds of active gas, which may include halogen based compound gas, can be adopted.

In addition, a cross-section of the treatment object 21 is just what it described in the first embodiment that even rectangles rather than a thing limited to a circle are preferable.

In addition, in a general idea of “the pot-shaped treatment object” in the fifth embodiment, it is included one end of a long-narrow tube in closed structure in addition to bottle shape as shown in FIG. 12.

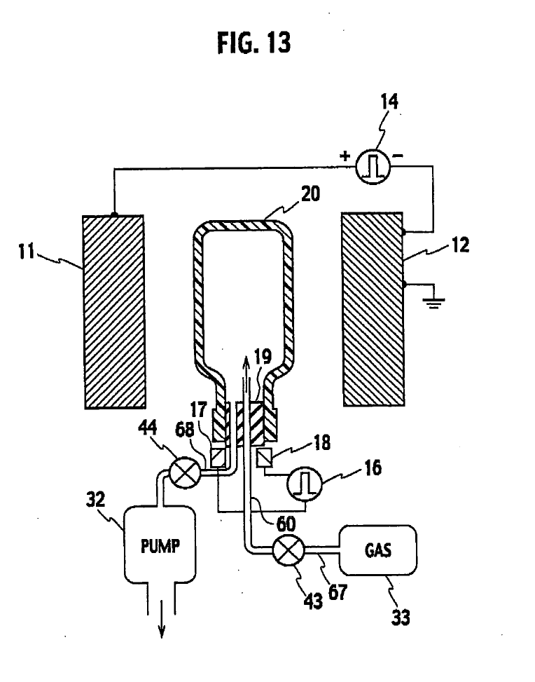

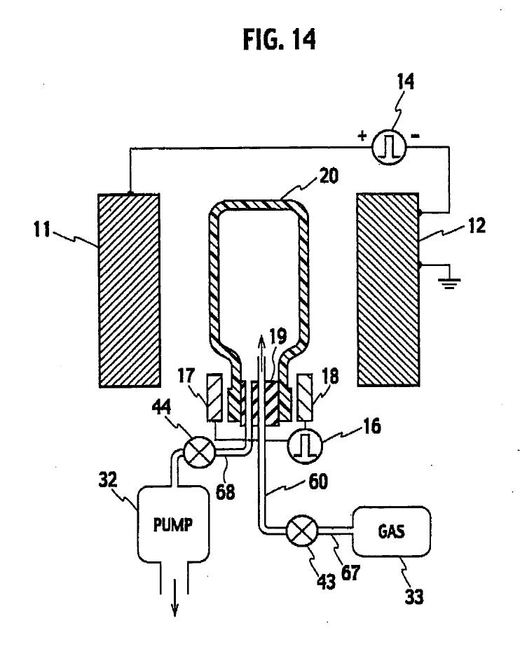

In FIG. 12, the first auxiliary electrode 17 to compose excited particle supplying system (16, 17 and 18) and the example which established the second auxiliary electrode 18 at the position that was not piled up in exhausting piping 68 were shown, the first auxiliary electrode 17 and the second auxiliary electrode 18 may he disposed at a position to catch both exhausting piping 68 and the feed piping 60 in as shown in FIG. 13. Furthermore, the first auxiliary electrode 17 and the second auxiliary electrode 18 may be disposed at a position sandwiching the neck adapter 19 as shown in FIG. 14. Furthermore, if initial plasma can be supplied after the flow of gas in the early stage an excited particle supplying system discharges electricity, and to start, the what may activate initial plasma by inductive plasma source rather than a thing limited to the parallel plate electrode configuration that seems to have always illustrated in FIG. 12 to FIG. 14 structure of others is similar for the case the surface treatment apparatus related to embodiment of the first and the third.

### Sixth Embodiment

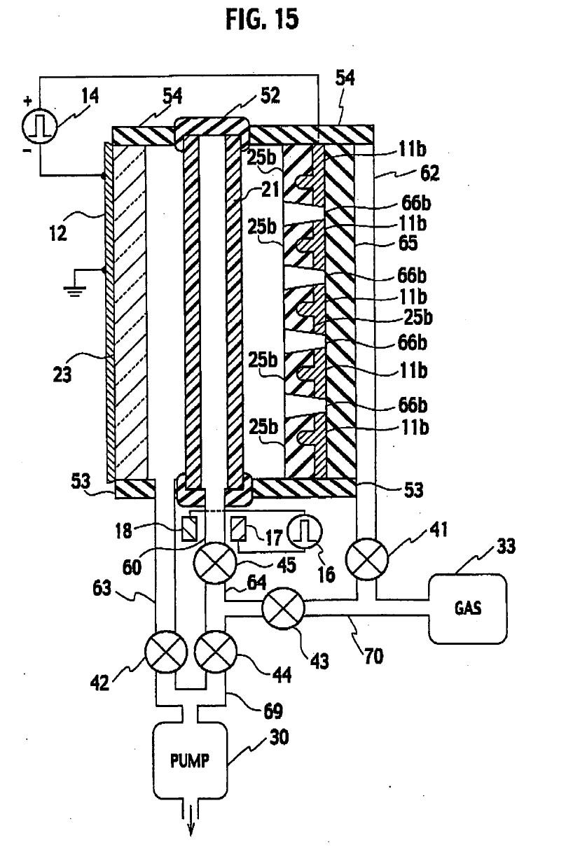

A surface treatment apparatus related to a sixth embodiment of the present invention embraces a vacuum manifold unit (43,44,45,60,64,69,70) to seal the process gas in by appointed processing pressure inside of the treatment object 21 from a dielectric as shown in FIG. 15 in another end of the treatment object 21 of full-fledged (FIG. 15, upper end) sealed tubular geometry (FIG. 15, bottom end).

The vacuum manifold unit (43,44,45,60,64,69,70) embraces an exhausting piping 69 connected to an injecting piping 70 and a first exhausting valve 44 connected to a first injecting valve 43 connected to a T-shaped piping 64 and the T-shaped piping 64 connected to a manifold valve 45 connected to the feed piping 60 connected with in another end of the treatment object 21 and the feed piping 60 and manifold valve 45 and the first exhausting valve 44 and the first injecting valve 43. The feed piping 60 is a piping made of dielectric material. Gas source 33 is connected to injecting piping 70, the vacuum pump 30 is connected to exhausting piping 69. Gas source 33 is a gas cylinder storing process gas. The first injecting valve 43 can adopt a needle valve that flow rate adjustment of gas is easy.

The process chamber (23, 53, 54, 62) is connected to the second injecting valve 41, and is connected with injecting piping 70, the process gas can seem to be supplied from the gas source 33 in the inside of the process chamber (23, 53, 54, 62), and it is provided. The process chamber (23, 53, 54, 62), so as to implement four planes of a rectangular parallelepiped, embraces a second electrode covering insulator (second main electrode covering insulator) 23 and the process chamber bottom lid 53, the chamber bottom lid 54 and the injection-adjusting chamber 62 Similar to the third embodiment, two side plates at a rearward portion of the paper (not illustrated) and at the near side (not illustrated) of the paper of FIG. 15, implement remaining two planes of the rectangular parallelepiped. There is no by the rectangular parallelepiped which is flatness, and the injection-adjusting chamber 62 embraces metallic five plane out of six planes of a rectangular parallelepiped, the gas supply layer 65 substitutes a single plane (a cross-sectional view shown in FIG. 15, the left side plane).

The second the exhausting piping 63 is connected to the process chamber (23, 53, 54, 62), the second exhausting valve 42 is connected to the second exhausting piping 63, the vacuum pump 30 is connected to the second exhausting valve 42 over exhausting piping 69. It is preferable for the first exhausting valve 44 and the second exhausting valve 42 to use the variable conductance valve through which the exhaust conductance can be adjusted.

At first, in the state that closed the first injecting valve 43, open manifold valve 45 arid the first exhausting valve 44, and a vacuum exhausts inside of the treatment object 21 in arrival pressure of about 10⁻¹ Pa to 10⁻⁶ Pa (background pressure) by vacuum pump 30.

The first the exhausting valve 44 is closed after arrival to the ultimate pressure, in the inside of the tubular treatment object 21, the first injecting valve 43, the T-shaped piping 64, the manifold valve 45 and the feed piping 60, process gas is supplied from the gas source 33 by opening the first injecting valve 43, and the process gas is supplied by another end side.

The treatment object 21 is near to an the atmospheric pressure of around 20-30 kPa, at the stage that arrived at processing pressure of less than or equal to an the atmospheric pressure, manifold valve 45 is closed, inside of the treatment object 21 is maintained in processing pressure.

On the other hand, injecting piping 70 and the second injecting valve 41, process gas is supplied, and, in the process chamber (23, 53, 54, 62), the process gas is supplied by constant flow rate in the ambient gas adjustment mechanism (62, 65, 66b, 26b) by gas source 33.

Similar to the third embodiment, the ambient gas adjustment mechanism (62, 65, 66b, 25b) embraces a the injection-adjusting chamber 62, gas supply layer 65 made of porous ceramics making the process gas from the injection-adjusting chamber 62 is distributed uniformly, a gas supply layer 65 as shown in FIG. 15, the first electrode protection layer (first main electrode protection layer) 25b having a plurality of gas supply holes 66b.

The ambient gas adjustment mechanism (62, 65, 66b, 25b) is implemented by a plurality of taper-shaped gas supply holes 66b penetrating through the first electrode protection layer (first main electrode protection layer) 25b, as shown in FIG. 7, the gas supply holes 66b are arranged in a form of two-dimensional matrix with a predetermined pitch. On the other hand, on the second electrode (second main electrode) 12, the second electrode covering insulator (second main electrode covering insulator) 23 of high purity quartz glasses is disposed.

Because of this it is supplied in the space that the process gas is made in the configuration of uniform shower by the ambient gas adjustment mechanism (62, 65, 66b, 25b), and surround the outside of internal the treatment object 21 of the process chamber (23, 53, 54, 62). The process gas supplied by the ambient gas adjustment mechanism (62, 65, 66b, 25b) is exhausted over the second exhausting piping 63 by the process chamber (23, 53, 54, 62).

Furthermore, the surface treatment apparatus related to the sixth embodiment is disposed by another end side of the treatment object 21, the active particle is poured into the process gas sealed in the discharge start early stage, the treatment object 21 excited particle supplying system to activate plasma (16,17,18) seems to be caught, and it is confronted each other, and is disposed, the first main electrode 11b composing a quasi-parallel plate electrode and initial plasma activated than the second main electrode 12 and injection of an activity particle are held, the pulse power supply 14 to apply an electric pulse (main pulse) to cause a plasma state inside of the treatment object 21 to between the first main electrode 11 and the second main electrode 12 is provided.

A plurality of T-shaped protrusions rather than flat slab configuration so as to implement the “quasi-parallel plate electrode” here same as embodiment of the second and the third the first main electrode 11b is arranged such that each of discharge points originates at each tips of the T-shaped protrusions. In this case, as for the first main electrode 11b, the periodical ladder-shaped electrode which is implemented by a plurality of bar (linear) electrodes to be being equal in price in parallel as had explained in the second embodiment, as a whole, the structure implemented by the first main electrode 11b and the second main electrode 12 is approximately “parallel plate electrode”.

In the chamber bottom lid 54, the top treatment object holder 52 holds one end (FIG. 15, upper end) side of the tubular treatment object 21, the bottom treatment object holder 51 holds another end of the treatment object 21 (FIG. 15, bottom end) in a sealing up state is connected to the chamber top lid 53.

If depending on materials of the treatment object 21, geometry and size, an appropriate change is added, and the structure that bottom treatment object holder 51 can be designed and manufactured with well-known gas joint or vacuum components is designed, it is preferable.

In FIG. 15, the second main electrode 12 is grounded so as to serve as the cathode, the case that high voltage is applied to the first main electrode 11b, and was used as an anode is illustrated, it turns over by polarity of pulse power supply 14 and is preferable in anode, the first main electrode 11b in the second main electrode 12 as the cathode. When the first main electrode 11b is assigned as the cathode, the first main electrode 11b is made into a slab-shaped electrode, and is grounded, the high voltage is applied as a type electrode to enjoy at a couple of the second main electrode 12, and the ambient gas adjustment mechanism (62, 65, 66b, 25b) is provided to the second main electrode 12.

The first auxiliary electrode 17 and an auxiliary pulse power supply to apply to the second auxiliary electrode 18 (although the illustration is omitted) are comprised in an electric pulse (a supporting pulse) to activate the first auxiliary electrode 17 the feed piping 60 of the treatment object 21 is caught similarly when the cross-section of the treatment object 21 described in the first embodiment in FIG. 15 excited particle supplying system that is just what it was described in embodiment of thing the first that is not a thing limited to a circle and the third (16,17,18), and implementing a parallel plate electrode and the second auxiliary electrode 18 and an activity particle.

If an active particle can be poured into the process gas that an excited particle supplying system is sealed in the discharge start early stage, the what may activate an activity particle by inductive plasma source rather than a thing limited to the parallel plate electrode configuration that seems to have always illustrated in FIG. 15 in what is preferable structure of others is similar for the case the surface treatment apparatus related to the first embodiment. After excitation of initial plasma by injection of an active particle, the surface treatment apparatus shown in FIG. 15 processes inside of the treatment object 21 of the tubular geometry that sealed one end by radicals included in plasma and the outside.

In the surface treatment apparatus related to the sixth embodiment, a high purity nitrogen gas can be supplied as the process gas, the “the process gas” is not always limited to nitrogen gas.

For example, for inside of the treatment object 21 and objects such as pasteurization or sterilization, mixed gas of nitrogen gas with various kinds of active gas such as halogen based compound gas can be adopted.

A high voltage pulse of the high repetition rate that seems to have been explained in the first embodiment is applied across the first main electrode 11 and the second main electrode 12 (See FIG. 4.)

FIG. 4A shows pulse width of 50-300 nano seconds preferable for the main pulse. When, in the surface treatment apparatus related to the sixth embodiment, if a distance between the first main electrode 11b and the second main electrodes, implementing a quasi-parallel plate electrode, is 15 millimeters, for the high voltage pulse with a repetition frequency of 2 kHz, a voltage value of around 24 kV is preferred.

Because a period is 500 microseconds, as shown in FIGS. 4A and 4B, at a repetition frequency of 2 k Hz for the high voltage pulse, the duty ratio becomes 0.3/500=0.006, non-thermal equilibrium low temperature plasma is generated efficiently and stably, without generating heat plasma ascribable to the high frequency discharge.

In the surface treatment apparatus related to the sixth embodiment, there are three operation modes explained in the third embodiment. That is to say a mode configured to ignite an discharge in mode making an discharge cause only in the inside of the treatment object 21, mode configured to ignite an discharge only at the outside of the treatment object 21, inside of the treatment object 21 and outside both tubular geometry that sealed one end, Similar to the third embodiment, those modes can be controlled by a pressure condition as shown by Eqs. (1)-(6).

The contents omit redundant explanation in what is similar to explanation in the third embodiment substantially.

### Seventh Embodiment

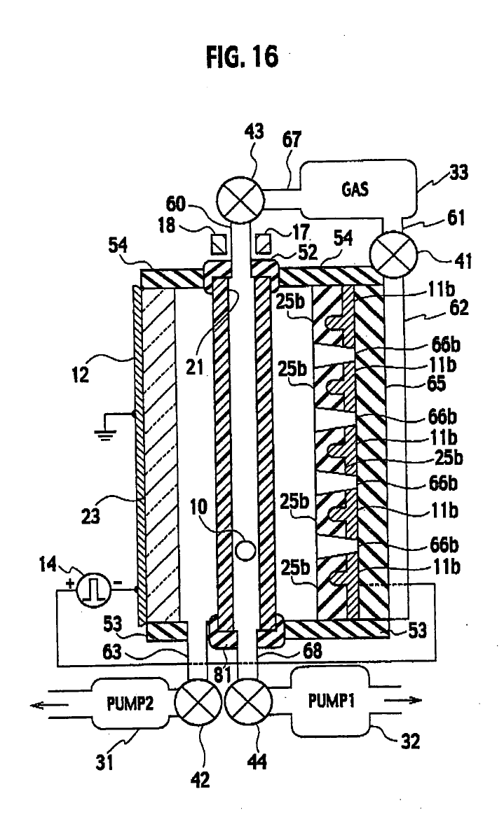

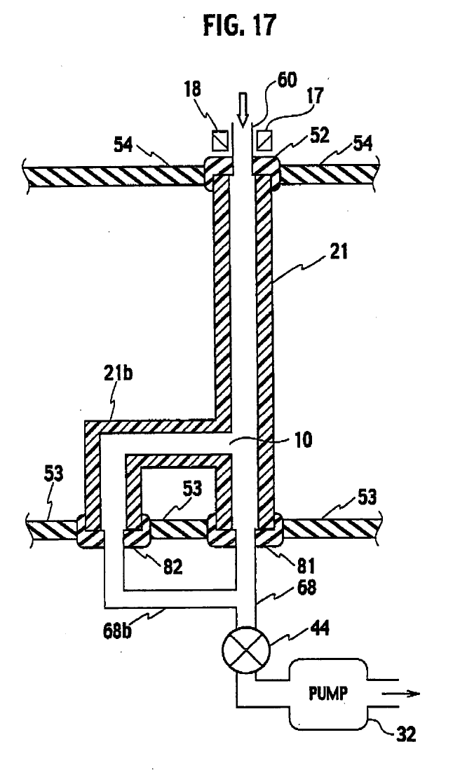

FIG. 16 and FIG. 17 are cross-sectional views looked at from a direction perpendicular each other. An endoscope may correspond to an example of “treatment object with tubular geometry with a branch”. A plurality of T-shaped protrusions, rather than flat slab configuration, implements the “quasi-parallel plate electrode”. Similar to the second, the third, the sixth embodiment the first main electrode 11b is arranged periodically, it is as an each of discharge points originates at each tips of the T-shaped protrusions, the structure as a whole is an approximately “parallel plate electrode”.

The second surface treatment apparatus related to the seventh embodiment goes to the second main electrode 12 side as the cathode in the process gas from the first main electrode 11b side as an anode. Similar to the third embodiment, the surface treatment apparatus related to the sixth embodiment, and it supplies in the shape of a shower, it further embraces a ambient gas adjustment mechanism (62, 65, 66b, 25b) to exhaust the process gas from the second exhausting piping 63 from the process chamber (23, 53, 54, 62).

The process chamber (23, 53, 54, 62), so as to implement four planes of a rectangular parallelepiped, embraces a second electrode covering insulator (second main electrode covering insulator) 23, a chamber top lid 53, the chamber bottom lid 54 and the injection-adjusting chamber 62, two side plates at a rearward portion of the paper (not illustrated) and at the near side (not illustrated) of the paper of FIG. 16, implement remaining two planes of the rectangular parallelepiped.

There is no by the rectangular parallelepiped which is flatness, and the injection-adjusting chamber 62 embraces metallic five plane out of six planes of a rectangular parallelepiped, the gas supply layer 65 substitutes a single plane (a cross-sectional view shown in FIG. 16, the left side plane).

To establish the sealed up space, the top treatment object holder 52 holds one end (upper-stream side) of the tubular treatment object 21 having the branched portion is provided to the chamber bottom lid 54. On the other hand, in the chamber top lid 53, tool for the branched portion pipe end maintenance 82 to hold an end of the branched portion pipe 21b branched off in the branched portion region 10 of bottom treatment object holder 81 to hold another end (down-stream side) of the treatment object 21 in a sealing up state as shown in FIG. 17 and the treatment object 21 in a sealing up state is provided.

Depending on materials, geometry and size of the treatment object 21, by applying required changes and modifications appropriately, the top treatment object holder 52, the bottom treatment object bolder 81 and the structure that the branched portion pipe end maintenance ingredient 82 can be designed and manufactured with well-known gas joint or vacuum components, easily.

The first exhausting piping 68 is connected to bottom treatment object holder 81, the branched portion exhaust piping 68b branched off the first exhausting piping 68 is connected to the branched portion pipe end maintenance ingredient 82.

And the first vacuum pump (first pump) 32 is connected to down-stream side of the first exhausting piping 68 over the first exhausting valve 44. By such a constitution, the first vacuum pump (first pump) 32 is connected to exhausting piping 68, the branched portion exhaust piping 68b and the first exhausting valve 44, and a vacuum can exhaust inside of the treatment object 21.

As shown in FIG. 16, the ambient gas adjustment mechanism (62, 65, 66b, 25b) embraces an the injection-adjusting chamber 62, a gas supply layer 65 made of porous ceramics making the process gas from the injection-adjusting chamber 62 is distributed uniformly, a gas supply layer 65, a first electrode protection layer (first main electrode protection layer) 25b having a plurality of gas supply holes 66b. The ambient gas adjustment mechanism (62, 65, 66b, 25b) is implemented by a plurality of taper-shaped gas supply holes 66b penetrating through electrode (first main electrode) protection layer 25b, and, Similar to the second embodiment, the gas supply holes 66b are arranged in a form of two-dimensional matrix with a predetermined pitch. (See FIG. 7.)

On the other hand, on the second electrode (second main electrode) 12, the second electrode covering insulator (second main electrode raw ring insulator) 23 of high purity quartz glasses is disposed. Furthermore, the surface treatment apparatus related to the seventh embodiment embraces a second the injecting valve 41 connected to the first injecting valve 43 and the second injecting piping 61 connected to the first injecting piping 67 connected to gas source 33 and a gas source 33 such as gas cylinders configured to store process gas and the second injecting piping 61 and the first injecting piping 67 as shown in FIG. 16. It is preferable to adopt a needle valve configured to adjust the flow rate for the first injecting valve 43, the second injecting valve 41.

The first injecting piping 67 and the first injecting valve 43, process gas is supplied from the gas source 33 in the inside of the tubular treatment object 21 having the branched portion, and the process gas is supplied by the upper-stream side, by vacuum pump (second pump) 31 that comprised downstream, the process gas drifts to the treatment object 21, the treatment object 21 is near in an the atmospheric pressure around 20-30 kPa, the pressure is kept at a processing pressure of less than or equal to an the atmospheric pressure.

On the other hand, in the process chamber (23, 53, 54, 62), the second injecting piping 61 and the second injecting valve 41, process gas is supplied from the gas source 33, and the flow of the process gas is shaped into the configuration of uniform shower by the ambient gas adjustment mechanism (62, 65, 66b, 25b). The process gas supplied by the ambient gas adjustment mechanism (62, 65, 66b, 25b) is exhausted by the second exhausting pining 63 from the process chamber (23, 53, 54, 62).

Then, as shown in FIG. 16 and FIG. 17, the second vacuum pump (second pump) 31 configured to evacuate space surrounding the outside of the treatment object 21, which is connected to the second exhausting piping 63 in the surface treatment apparatus related to the seventh embodiment.

The second vacuum pump (second pump) 31 is connected to the second exhausting piping 63 and the second exhausting connected to the process chamber (23, 53, 54, 62). It is preferable for the first exhausting valve 44 and the second exhausting valve 42 to use the variable conductance valve through which the exhaust conductance can be adjusted.

In FIG. 16, the second main electrode 12 is grounded so as to serve as the cathode, the case that high voltage is applied to the main electrode 11b, and was used as an anode is illustrated, it turns over by polarity of pulse power supply 14 and is preferable in anode, the first main electrode 11b in the second main electrode 12 as the cathode. When the first main electrode 11b is assigned as the cathode, the first main electrode 11b is made into a slab-shaped electrode, and is grounded, the high voltage is applied as a type electrode to enjoy at a couple of the second main electrode 12, and the ambient gas adjustment mechanism (62, 65, 66b, 25b) is provided to the second main electrode 12.

Similar to the first embodiment, a narrow tube having an inside diameter of less than or equal to 7-5 millimeters can process the length of a long-narrow tube that length of a tubular geometry part aside from the branched portion (trunk portion) is more than 4-7 meters may serve as the tubular treatment object 21 having the branched portion in the surface treatment apparatus related to the seventh embodiment as well, even if length of the trunk portion is less than 4 meters, more than 7 millimeters inside diameter, the treatment object 21 can be processed. In addition, a cross-section of the treatment object 21 is just what it described in the first embodiment not to be a branched pipe and the thing, which it is circular, and is, limited both of the trunk portion.

The first auxiliary electrode 17 and an auxiliary pulse power supply to apply to the second auxiliary electrode 18 (although the illustration is omitted) are comprised in an electric pulse to cause FIG. 16 excited particle supplying system (17,18) and the first auxiliary electrode 17 so as to sandwich the feed piping 60 connected to the upper-stream side of the treatment object 21 is caught similarly when it was described in the first embodiment in FIG. 17, and implementing a parallel plate electrode so as to generate initial plasma (a supporting pulse).

The feed piping 60 is a piping made of dielectric material. If initial plasma can be supplied after the flow of gas in the early stage an excited particle supplying system discharges electricity, and to start, the what may activate initial plasma by inductive plasma source rather than a thing limited to the parallel plate electrode configuration that seems to have always illustrated in FIG. 16 and FIG. 17 structure of others is similar for the case the surface treatment apparatus related to the first embodiment.

After excitation of initial plasma, FIG. 16 and the surface treatment apparatus shown in FIG. 17 process inside of the tubular treatment object 21 having the branched portion and the outside by radicals included in plasma. In the surface treatment apparatus related to the seventh embodiment, a high purity nitrogen gas can be supplied as the process gas in the treatment object 21 from the upper-stream side, the “the process gas” is not always limited to nitrogen gas. For example, for inside of the treatment object 21 and objects such as pasteurization or sterilization, mixed gas of nitrogen gas with various kinds of active gas such as halogen based compound gas can be adopted.

A high voltage pulse of the high repetition rate that seems to have been explained in the first embodiment is applied across the first main electrode 11 and the second main electrode 12 (See FIG. 4.)

When, in the surface treatment apparatus related to the seventh embodiment, if a distance between the first main electrode 11b and the second main electrodes, implementing a quasi-parallel plate electrode, is 15 millimeters, for the high voltage pulse with a repetition frequency of 2 kHz, a voltage value of around 24 kV is preferred. A period is 500 microseconds, and, in the case of repetition frequency 2 kHz of the high voltage pulse, the duty ratio becomes 0.3/500=0.006 repeatedly.

Because of this it is generated the efficiency stability non-thermal equilibrium low temperature plasma, without generating heat plasma ascribable to the high frequency discharge. In the surface treatment apparatus related to the seventh embodiment, there are three operation modes explained in the third embodiment. That is to say a first mode configured to ignite an discharge in the inside of the treatment object 21, a second mode configured to ignite an discharge only at the outside of the treatment object 21, a third mode configured to ignite an discharge both inside and outside of the treatment object 21 with tubular geometry. Therefore, similar to the third embodiment, those modes can be controlled by pressure conditions as shown by Eqs. (1)-(6).

The contents omit redundant explanation in what is similar to explanation in the third embodiment substantially.

### Eighth Embodiment

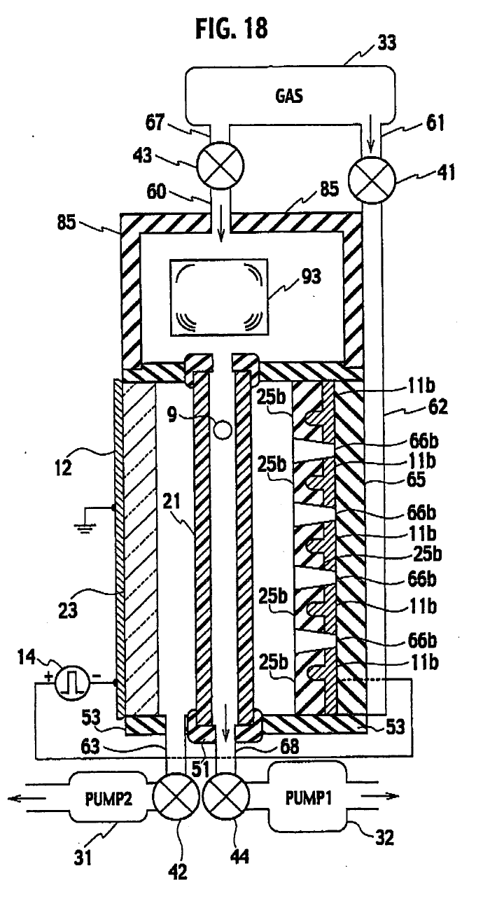

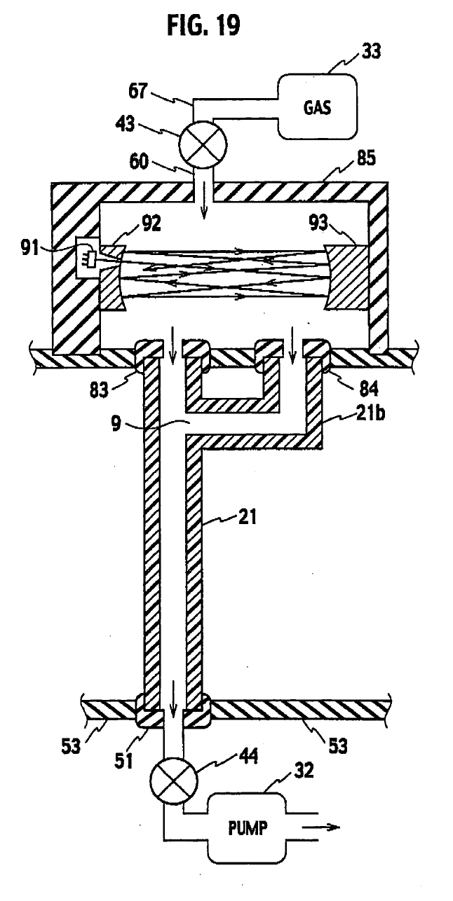

FIG. 18 and FIG. 19 are cress-sectional views looked at from a direction perpendicular each other. As an example of “treatment object with tubular geometry with a branch”, an endoscope described in the seventh embodiment falls, the topology that reversed the upper-stream side and down-stream side of an endoscope of the seventh embodiment is just coped with.

A plurality of T-shaped protrusions rather than flat slab configuration so as to implement the “quasi-parallel plate electrode”. Similar to the second, third, sixth, and seventh embodiments the first main electrode 11b is arranged periodically, it is as an each of discharge points originates at each tips of the T-shaped protrusions, the structure as a whole is an approximately “parallel plate electrode”.

The first reflecting mirror 92 that comprised, for example, in excited particle generation chamber 85 and this excited particle generation chamber 85 as shown in FIG. 18 and FIG. 19 excited particle supplying system (85,91,92,93) and the second reflecting mirror 93 and ultraviolet rays irradiation mechanism 91 are comprised. The first reflecting mirror 92 is a concave lens of hole autumn having a through-hole to irradiate ultraviolet rays in one part. And the second reflecting mirror 93 is a concave lens ultraviolet rays the first introduced from a through-hole of the first reflecting mirror 92 by the first reflecting mirror 92 and the second reflecting mirror 93 are faced, and disposing reflects back interlocking between reflecting mirror 92 and the second reflecting mirror 93, and the process gas supplied in excited particle generation chamber 85 is activated, the excitation particle is generated.

As ultraviolet rays irradiation mechanism 91, semiconductor emission of light elements such as a GaN based compound semiconductor, a ZnSe based compound semiconductor, a ZnO based compound semiconductor, a semiconductor laser with the use of a wideband gap semiconductor of SiC based compound semiconductors or light emitting diode are desirable for miniaturization.

However, even another solid laser is how preferable even a gas laser emitting light by ultraviolet rays of excimer laser. When large-scale ultraviolet rays irradiation mechanism 91 of gas lasers of excimer laser is used, the of particle generation room 85 to activate ultraviolet rays irradiation mechanism 91, it disposes outside, the window materials transmitting by ultraviolet rays of sapphire, process gas is supplied and, the of excited particle generation chamber 85, the if ultraviolet rays are introduced inside.

In this way the first ultraviolet rays from ultraviolet rays irradiation mechanism 91 disposed outside of exerted particle generation chamber 85 are introduced between the first reflecting mirror 92 and the second reflecting mirror 93 from a through-hole of reflecting mirror 92, interlocking is reflected back between the first reflecting mirror 92 and the second reflecting mirror 93, and the process gas can be activated.

The second surface treatment apparatus related to the eighth embodiment goes to the second main electrode 12 side as the cathode in the process gas from the first main electrode 11b side as an anode. Similar to the third, the sixth, the surface treatment apparatus related to the seventh embodiment, and it supplies in the shape of a shower, it further embraces a ambient gas adjustment mechanism (62, 65, 66b, 25b) to exhaust the process gas from the second exhausting piping 63 from the process chamber (23, 53, 54, 62). The process chamber (23, 53, 54, 62), so as to implement four planes of a rectangular parallelepiped, embraces a second electrode covering insulator (second main electrode covering insulator) 23, a chamber top lid 53, the chamber bottom lid 54 and the injection-adjusting chamber 62, two side plates at a rearward portion of the paper (not illustrated) and at the near side (not illustrated) of the paper of FIG. 18, implement remaining two planes of the rectangular parallelepiped.

There is no by the rectangular parallelepiped which is flatness, and the injection-adjusting chamber 62 embraces metallic five plane out of six planes of a rectangular parallelepiped, the gas supply layer 65 substitutes a single plane (a cross-sectional view shown in FIG. 18, the left side plane).

As shown in FIG. 19, tool for the branched portion pipe end maintenance 84 to hold an end of the branched portion pipe 21b branched off the trunk portion in top treatment object holder 83 to hold in a full-fledged (upper-stream side) sealing up state and the branched portion region 9 of the trunk portion of the treatment object 21 in a sealing up state is provided in the chamber bottom lid 54. With top treatment object holder 83 and the branched portion pipe end maintenance ingredient 84, an aperture is established in a bottom of excited particle generation chamber 85 to compose excited particle supplying system (85,91,92,93), is connected to excited particle generation chamber 85. On the other hand, in the chamber top lid 53, bottom treatment object holder 51 holds another end (down-stream side) of the treatment object 21 in a sealing up state as shown in FIG. 19 is arranged.

Depending on materials, geometry and size of the treatment object 21, by applying required changes and modifications appropriately, and top treatment object holder 83, the branched portion pipe end maintenance ingredient 84 and the structure that bottom treatment object holder 51 can be designed and manufactured with well-known gas joint or vacuum components, easily.

The first exhausting piping 68 is connected to bottom treatment object holder 51. And the first vacuum pump (first pump) 32 is connected to down-stream side of the first exhausting piping 68 over the first exhausting valve 44. By such a constitution, the first vacuum pump (first pump) 32 is connected to exhausting piping 68 and the first exhausting valve 44, and a vacuum can exhaust inside of the treatment object 21.

The ambient gas adjustment mechanism (62, 65, 66b, 25b) embraces a the injection-adjusting chamber 62, gas supply layer 65 made of porous ceramics making the process gas from the injection-adjusting chamber 62 is distributed uniformly, a gas supply layer 65 as shown in FIG. 18, the first electrode protection layer (first main electrode protection layer) 25b having a plurality of gas supply holes 66b. The ambient gas adjustment mechanism (62, 65, 66b, 25b) is implemented by a plurality of taper-shaped gas supply holes 66b penetrating through the first electrode protection layer (first main electrode protection layer) 25b, and, Similar to the second embodiment, the gas supply holes 66b are arranged in a form of two-dimensional matrix with a predetermined pitch. (See FIG. 7.). On the other hand, on the second electrode (second main electrode) 12, the second electrode covering insulator (second main electrode covering insulator) 23 of high purity quartz glasses is disposed.

Furthermore, the surface treatment apparatus related to the eighth embodiment embraces a second the injecting valve 41 connected to the first injecting valve 43 and the second injecting piping 61 connected to the first injecting piping 67 connected to gas source 33 and a gas source 33 such as gas cylinders configured to store process gas and the second injecting piping 61 and the first injecting paring 67 as shown in FIG. 18. It is preferable to adopt a needle valve configured to adjust the flow rate for the first injecting valve 43, the second injecting valve 41. The first injecting valve 43 is connected to the feed piping 60, the feed piping 60 is connected to a ceiling part of excited particle generation chamber 85. In the surface treatment apparatus related to the eighth embodiment, the feed piping 60 does not have to be always piping made of dielectric material.

In the inside of excited particle generation chamber 85, the first injecting piping 67, the first injecting valve 43 and the feed piping 60, process gas is supplied from the gas source 33, and the process gas is supplied by the upper-stream side.

The process gas supplied inside of excited particle generation chamber 85 goes through an aperture of top treatment object holder 83 and tool for the branched portion pipe end maintenance 84 that is inserted in a bottom of excited particle generation chamber 85, and it is supplied in the trunk portion of each the treatment object 21 and the branched portion pipe 21b.

At this chance, in the inside of excited particle generation chamber 85, excited particles are generated, the a generated excitation particle goes through top treatment object holder 83 and tool for the branched portion pipe end maintenance 84 of a bottom of excited particle generation chamber 85 along with the process gas, and is poured into the trunk portion of each the treatment object 21 and the branched portion pipe 21b, initial plasma is generated in the inside of the trunk portion of the treatment object 21 and inside of the branched portion pipe 21b.

The process gas supplied in the trunk portion of the treatment object 21 and the branched portion pipe 21b is exhausted after junction in branching site 9 by vacuum pump (second pump) 31 that comprised downstream of the treatment object 21, the treatment object 21 is near in an the atmospheric pressure of around 20-30 kPa, the pressure is kept at a processing pressure of less than or equal to an the atmospheric pressure.

On the other hand, in the process chamber (23, 53, 54, 62), the second injecting piping 61 and the second injecting valve 41, process gas is supplied from the gas source 33, and the flow of the process gas is shaped into the configuration of uniform shower by the ambient gas adjustment mechanism (62, 65, 66b, 25b). The process gas supplied by the ambient gas adjustment mechanism (62, 65, 66b, 25b) is exhausted by the second exhausting piping 63 from the process chamber (23, 53, 54, 62).

Then, as shown in FIG. 18 and FIG. 19, the second vacuum pump (second pump) 31 configured to evacuate space surrounding the outside of the treatment object 21, which is connected to the second exhausting piping 63 in the surface treatment apparatus related to the eighth embodiment. The second vacuum pump (second pump) 31 is connected to the second exhausting piping 63 and the second exhausting valve 42, is connected to the process chamber (23, 53, 54, 62). It is preferable for the first exhausting valve 44 and the second exhausting valve 42 to use the variable conductance valve through which the exhaust conductance can be adjusted.

In FIG. 18, the second main electrode 12 is grounded so as to serve as the cathode, the case that high voltage is applied to the first main electrode 11b, and was used as an anode is illustrated, it turns over by polarity of pulse power supply 14 and is preferable in anode, the first main electrode 11b in the second main electrode 12 as the cathode. When the first main electrode 11b is assigned as the cathode, the first main electrode 11b is made into a slab-shaped electrode, and is grounded, the high voltage is applied as a type electrode to enjoy at a couple of the second main electrode 12, and the ambient gas adjustment mechanism (62, 65, 66b, 25b) is provided to the second main electrode 12.

Similar to the first embodiment, a narrow tube having an inside diameter of less than or equal to 7-5 millimeters can process the length of a long-narrow tube that length of a tubular geometry part aside from the branched portion (trunk portion) is more than 4-7 meters may serve as the tubular treatment object 21 having the branched portion in the surface treatment apparatus related to the eighth embodiment as well, even if length of the trunk portion is less than 4 meters, more than 7 millimeters inside diameter, the treatment object 21 can he processed.

In addition, a cross-section of the treatment object 21 is just what it described in the first embodiment not to be a branched pipe and the thing, which it is circular, and is, limited both of the trunk portion.

After excitation of initial plasma, as for FIG. 18 and the surface treatment apparatus shown in FIG. 19, inside is processed by radicals included in plasma drifting to inside of the tubular treatment object 21 having the branched portion by uniformity flow rate. In addition, the outside of the tubular treatment object 21 having the branched portion is processed by radicals included in plasma generated outside of treatment object.

In the surface treatment apparatus related to the eighth embodiment, a high purity nitrogen gas can be supplied as the process gas in the inside of the treatment object 21 and the outside, the “the process gas” is not always limited to nitrogen gas.

For example, for inside of the treatment object 21 and objects such as pasteurization or sterilization, mixed gas of nitrogen gas with various kinds of active gas such as halogen based compound gas can be adopted.

A high voltage pulse of the high repetition rate that seems to have been explained in the first embodiment is applied across the first main electrode 11 and the second main electrode 12 (See FIG. 4.)

When, in the surface treatment apparatus related to the eighth embodiment, if a distance between the first main electrode 11b and the second main electrodes, implementing a quasi-parallel plate electrode, is 15 millimeters, for the high voltage pulse with a repetition frequency of 2 kHz, a voltage value of around 24 kV is preferred. A period is 500 microseconds, and, in the case of repetition frequency 2 kHz of the high voltage pulse, the duty ratio becomes 0.3/500=0.006 repeatedly. Because of this it is generated the efficiency stability non-thermal equilibrium low temperature plasma, without generating heat plasma ascribable to the high frequency discharge.

In the surface treatment apparatus related to the eighth embodiment, there are three operation modes explained in the third embodiment. That is to say a first mode configured to ignite an discharge only in the inside of the treatment object 21, a second mode configured to ignite an discharge only at the outside of the treatment object 21, a third mode configured to ignite both inside and outside of the treatment object 21 having tubular geometry with a branch. Similar to the third embodiment, those modes can be controlled by a pressure condition, as shown by Eqs. (1)-(6). The contents omit redundant explanation in what is similar to explanation in the third embodiment substantially.

### Ninth Embodiment

In the third, sixth to eighth embodiments, examples to control three operation modes by a pressure condition in the surface treatment apparatus is explained by Eqs. (1)-(6). A first mode configured to ignite an discharge only in the inside of the treatment object 21, a second mode configured to ignite an discharge only at the outside of the treatment object 21, a third mode configured to ignite both inside and outside of the treatment object 21 are controlled by choosing a pressure condition as shown in an Eqs. (1)-(6). That is to say, even if control of three operation modes uses a parameter aside from pressure of the process gas, it can control. It is temperature of the process gas which one example of other parameters explains in the surface treatment apparatus related to the ninth embodiment of the present invention.

In addition, even a point to comprise the second injecting valve 41 connected to the first injecting valve 43 and the second injecting piping 61 connected to the first injecting piping 67 connected to gas source 33 and a gas source 33 such as gas cylinders configured to stare process gas and the second injecting piping 61 and the first injecting piping 67 is similar to the surface treatment apparatus related to the third embodiment.

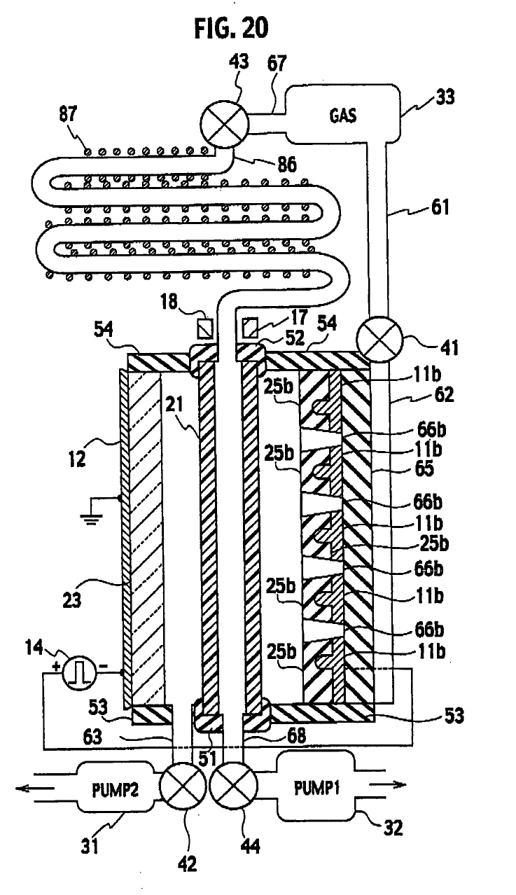

However, the first surface treatment apparatus related to the ninth embodiment of the present invention is different from the surface treatment apparatus related to the third embodiment in the feed pining 86 connected with in injecting valve 43 at a point comprising pre-beater 87 as shown in FIG. 20. It is desirable to get constant application of heat distance by the topology that the feed piping 86 meanders through in the shape of meandering line as shown in FIG. 20 in order to raise the application of heat efficiency of the process gas.

The feed piping 86 does not have to be always piping made of dielectric material, a disposed point excited particle supplying system (17,18) consists of a dielectric. The first injecting piping 67 and the first injecting valve 43, process gas is supplied from the gas source 33 in the inside of the tubular treatment object 21, and the process gas is supplied by the upper stream side, by vacuum pump (second pump) 31 that comprised downstream, the process gas drifts to the treatment object 21, the treatment object 21 is kept by appointed pressure, the when a mode configured to ignite an discharge among three operation modes only in the inside of the treatment object 21 is chosen, because the ambient gas adjustment, mechanism (62, 65, 66b, 25b) is provided, and 30-50 degrees Celsius lift temperature of the process gas drifting to inside of the treatment object 21 by what is energized in pre-heater 87 than temperature of the process gas drifting outside of the treatment object 21, an discharge is easy to be generated only in the inside of the treatment object 21, and it can be done.

Of course gas pressure P1 of the treatment object 21 inside is made around 10-40 kPa in the inside of the treatment object 21 in order to be caused, and it is desirable to lower than outside gas pressure P2 of the treatment object 21.

In addition, it is desirable to more extremely than, the atmospheric pressure P3 only lower outside gas pressure P2 of the treatment object 21 to around 80-90 kPa with the atmospheric pressure P3=101 kPa so that Eq. (1) shows whether it is equal, the a mode configured to ignite an discharge surely more stably only in the inside of the treatment object 21 as well because 30-50 degrees Celsius lift temperature of the process gas drifting to inside of the treatment object 21 than temperature of the process gas drifting outside of the treatment object 21 can be chosen.

In addition, when outside gas pressure P2 of the treatment object 21 is near to gas pressure P1 of the treatment object 21 inside, even if it is put, the a mode configured to ignite an discharge only in the inside of the treatment object 21 can be chosen.

The process chamber (23, 53, 54, 62) and structure of the ambient gas adjustment mechanism (62, 65, 66b, 25b) omit redundant explanation in what is similar to the surface treatment apparatus related to the third embodiment. In addition, though the Although the illustration is omitted, the a buried heater is established in the inside of the ambient gas adjustment mechanism (62, 65, 66b, 26b), and temperature of the process gas flowing outside of the treatment object 21 is raised than temperature of the process gas drifting to inside of the treatment object 21, and a mode configured to ignite an discharge only at the outside of the treatment object 21 can be chosen.

In addition, a Peltier cooling unit is provided to inside of the ambient gas adjustment mechanism (62, 65, 66b, 25b), and, by electronic cooling (Peltier effect), temperature of the process gas drifting outside of the treatment object 21 is done lower than temperature of the process gas drifting to inside of the treatment object 21, and an discharge is controlled only at the outside of the treatment object 21, the discharge is waked up in the inside of the treatment object 21.

Instead of a Peltier cooling unit, piping of refrigerant gas is provided to inside of the ambient gas adjustment mechanism (62, 65, 66b, 25b), and temperature of the process gas flowing outside of the treatment object 21 is done lower than temperature of the process gas drifting to inside of the treatment object 21, and an discharge can be controlled only at the outside of the treatment object 21.

Others omit redundant explanation in what is similar to the surface treatment apparatus related to the third embodiment substantially.

### Tenth Embodiment

As explained in the ninth embodiment, the control of three operation modes can be controlled by mechanism of a parameter aside from pressure of the process gas. One example of other parameters is temperature of the process gas to explain in the surface treatment apparatus related to the ninth embodiment of the present invention, by a method to introduce trigger gas doing an discharge easily into only an discharge point desired in the discharge early stage, three operation modes can be controlled.

However, as for the first surface treatment apparatus related to the tenth embodiment of the present invention, the first T-shaped piping 67t to introduce trigger gas into injecting valve 43c are connected to, at the point where the second T-shaped piping 61t to introduce trigger gas into the second injecting valve 41c are connected to, it is different from the surface treatment apparatus related to the third embodiment.

Furthermore, the first branching site of the first T-shaped piping 67t is connected to trigger gas introduction valve 43b and the first trigger gas introduction piping 67b, is connected to the first trigger gas source 88a. In addition, the second branching site of the second T-shaped piping 61t is connected to trigger gas introduction valve 41b and the second trigger gas introduction piping 61b, is connected to the second trigger gas source 88b.

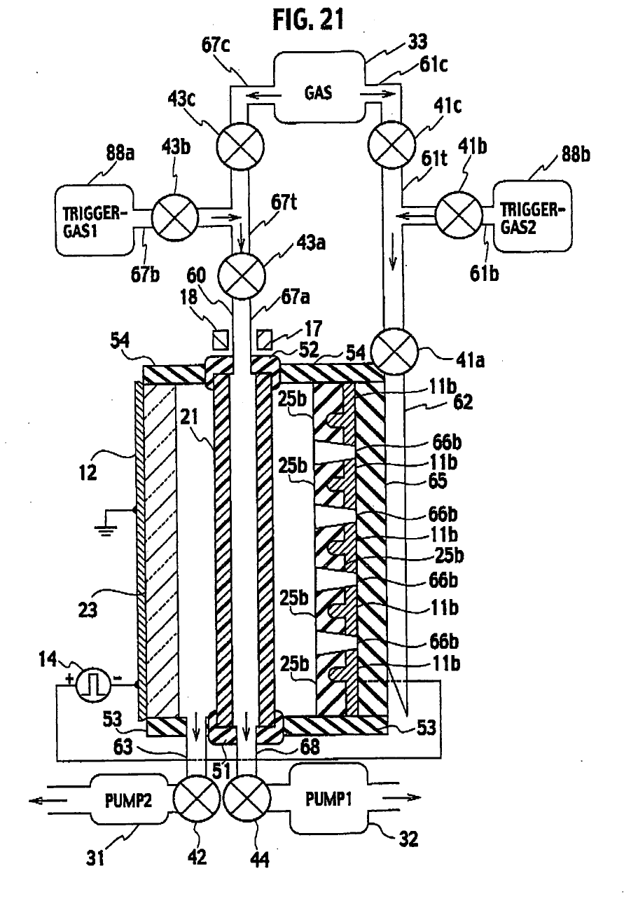

The first trigger gas source 88a and the second trigger gas source 88b illustrates in FIG. 21 as another gas source, the even common gas source can be employed. The first trigger gas source 88a and the second trigger gas source 88b is the cylinder which easy gas was filled with by discharge such as helium (Ha), Argon (Ar).

The first trigger gas introduction valve 43b and the valve that response time such as an electromagnetic valve or an air pressure valve (a response) is fast as for the second trigger gas introduction valve 41b are preferable. Furthermore, the first down stream side of the first T-shaped piping 67t is connected to the feed piping 60 over manifold valve 43a. The feed piping 60 is a piping made of dielectric material. On the other hand, the second down stream side of the second T-shaped piping 61t is connected to the ambient gas adjustment mechanism (62, 65, 66b, 25b) over manifold valve 41a.

The first injecting piping 67c, the first injecting valve 43c, the first T-shaped piping 67t, the first manifold valve 43a and the feed piping 60, process gas is supplied from the gas source 33 in the inside of the tubular treatment object 21, the process gas is supplied by the upper-stream side, by vacuum pump (second pump) 31 that comprised downstream, the process gas drifts to the treatment object 21, the treatment object 21 is kept by appointed pressure. At this chance it is put at the beginning of an discharge, and a short time, the first trigger gas introduction valve 43b are thrown open when a mode making raise an discharge only in the inside of the treatment object 21 is chosen among three operation modes, the trigger gas flows from the first trigger gas source 88a, the T-shaped piping 67t, the first manifold valve 43a and the feed piping 60, process gas is supplied, and, by what is introduced in the inside of the treatment object 21, an discharge is easy to be generated only in the inside of the treatment object 21, and this the first trigger gas can be done.

Of course gas pressure P1 of the treatment object 21 inside is made around 10-40 kPa in the inside of the treatment object 21 in order to be caused, and it is desirable to lower than outside gas pressure P2 of the treatment object 21. In addition, it is equal with the atmospheric pressure P3=101 kPa in outside gas pressure P2 of the treatment object 21 so that an Eq. (1) shows or, the more extremely than the atmospheric pressure P3 preferred will lower around 80-90 kPa, the a second, a mode making it is pulse-like, and ignite an discharge by what it is introduced into surely more stably only in the inside of the treatment object 21 as well can be chosen in trigger gas. In addition, when outside gas pressure P2 of the treatment object 21 is near to gas pressure P1 of the treatment object 21 inside, even if it is put, the a mode configured to ignite an discharge only in the inside of the treatment object 21 by introducing trigger gas can be chosen.

On the other hand, the second injecting piping 61c, the second injecting valve 41c, the second T-shaped piping 61t, the second manifold valve 41a, process gas is supplied from the gas source 33 in the ambient gas adjustment mechanism (62, 65, 66b, 25b), and the process gas is supplied by the upper-stream side, by vacuum pump (second pump) 31 that comprised downstream, the process gas drifts to a the process chamber (23, 53, 54, 62), the a the process chamber (23, 53, 54, 62) is kept by appointed pressure.

At this chance it is put at the beginning of an discharge, and a short time, the second trigger gas introduction valve 41b are thrown open when a mode making raise an discharge only at the outside of the treatment object 21 is chosen among three operation modes, the trigger gas flows from the second trigger gas source 88b, the T-shaped piping 61t, the second manifold valve 41a, process gas is supplied, and, by what is introduced in the inside of the process chamber (23, 53, 54, 62), an discharge is easy to be generated only at the outside of the treatment object 21, and this the second trigger gas can be done.

Of course in order to make an discharge cause only at the outside of the treatment object 21, the preferred will set in a pressure condition as shown by Eq. (3), (4), (5) or (6), the a second, a mode making it is pulse-like, and ignite an discharge by what it is introduced into surely more stably only in the inside of the treatment object 21 as well can be chosen in trigger gas. In addition, when outside gas pressure P2 of the treatment object 21 is near to gas pressure P1 of the treatment object 21 inside, even if it is put, the a mode configured to ignite an discharge only at the outside of the treatment object 21 by introducing trigger gas can be chosen.

About a mode making an discharge cause in the inside and outside both the treatment object 21, trigger gas flows into both, the trigger gas flows. In addition, it is made the condition that is hard to discharge one, and it may make trigger gas flow in there. For example, other constitution, the process chamber (23, 53, 54, 62) and structure of the ambient gas adjustment mechanism (62, 65, 66b, 25b) omit redundant explanation in what is similar to the surface treatment apparatus related to the third embodiment.

### Eleventh Embodiment

As shown in FIG. 22, a surface treatment apparatus related to a eleventh embodiment of the present invention apparatus encompasses a dielectric housing (74, 75 and 76) configured to accommodate an treatment object 5; a dielectric housing (74, 75 and 76) configured to accommodate an treatment object 5; a vacuum evacuating system (32, 44 and 68) configured to evacuate a process gas introduced at a specific flow rate from an introducing piping provided at other end of the dielectric housing (74, 75 and 76) having one end dosed, from an exhaust piping provided at the other end, and maintaining the pressure of the process gas inside the dielectric housing (74, 75 and 76) at a process pressure; an excited particle supplying system (16,17 and 18) disposed at the gas supply upstream side to the dielectric housing (74, 75 and 76), configured to supply exerted particles for inducing initial discharge in a main body of the dielectric housing (74, 75 and 76); and a first main electrode 11 and a second main electrode 12 disposed oppositely to each other, defining a treating region of the treatment object as a main plasma generating region disposed therebetween, wherein the excited particle supplying system (16,17 and 18) is driven at least until generation of main plasma, and main pulse of duty ratio of 10⁻⁷ to 10⁻¹ is applied between the first main electrode 11 and second main electrode 12, to generate a non-thermal equilibrium plasma flow inside the dielectric housing (74, 75 and 76), and thereby a surface of the treatment object 5 is treated.

The dielectric housing (74, 75 and 76) is implemented by a dielectric tube 74 and a dielectric flange plate 75. The dielectric tube 74 and the dielectric flange plate 75 is sealed by o-ring 76 so as to establish a vacuum tight structure. On the second main electrode 12, a second main electrode covering insulating film 77 is disposed so as to cover the surface of the second main electrode 12, and the dielectric housing (74, 75 and 76) is fixed on the second main electrode covering insulating film 77.

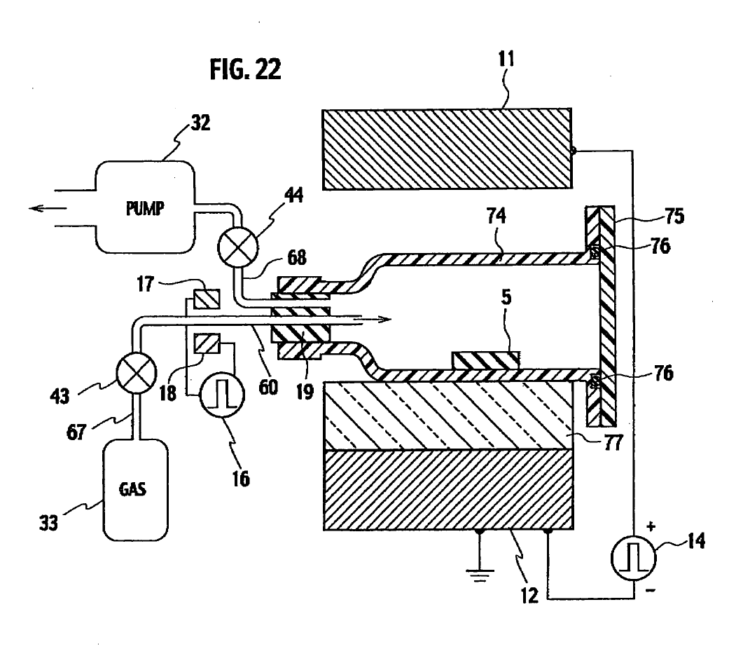

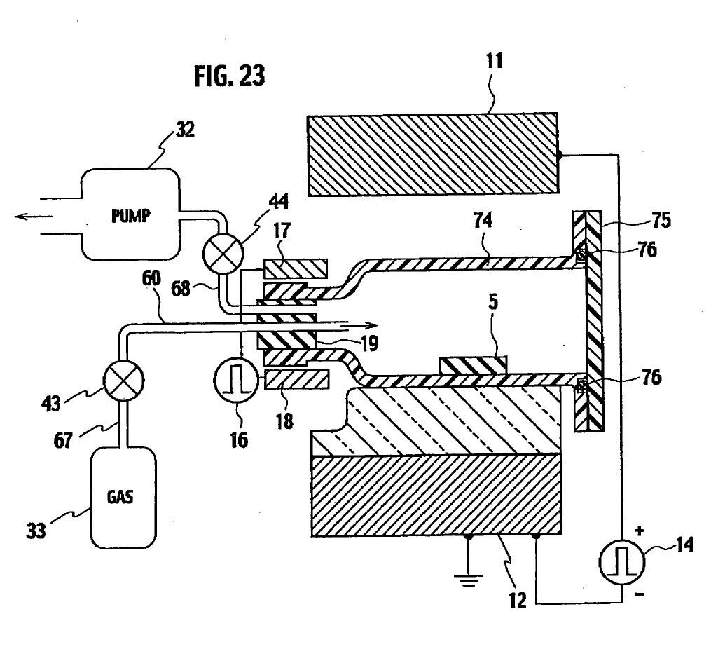

In FIG. 22, the first auxiliary electrode 17 and the second auxiliary electrode 18, implementing the excited particle supplying system (16, 17 and 18), are arranged at a position where the feed piping 60 does not overlap with position of the exhausting piping 68 were shown. However, the first auxiliary electrode 17 and the second auxiliary electrode 18 may be disposed at a position to sandwich both the exhausting piping 68 and the feed piping 60 as shown in FIG. 23. FIG. 23 is a cross-sectional view schematically explaining essential structure of the surface treatment apparatus in accordance with a first modification of the eleventh embodiment of the present invention.

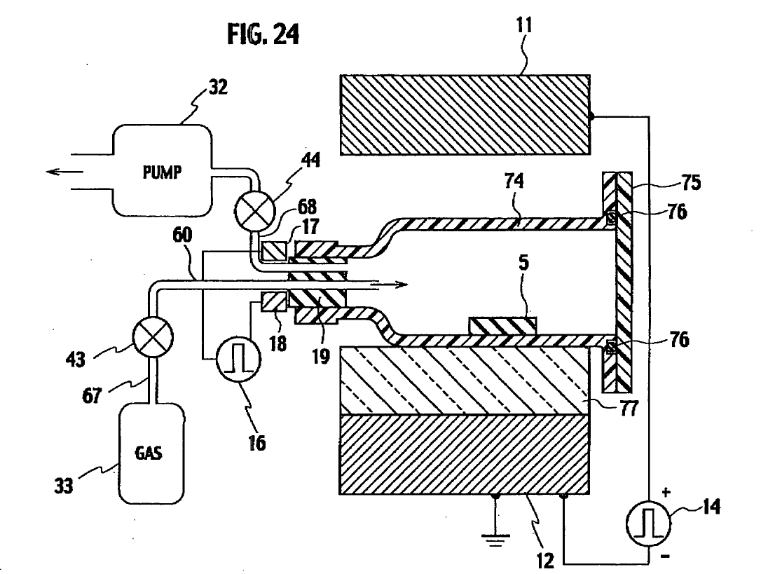

Furthermore, the first auxiliary electrode 17 and the second auxiliary electrode 18 may be disposed at a position sandwiching the neck adapter 19 as shown in FIG. 24. FIG. 24 is a cross-sectional view schematically explaining essential structure of the surface treatment apparatus in accordance with a second modification of the eleventh embodiment of the present invention.

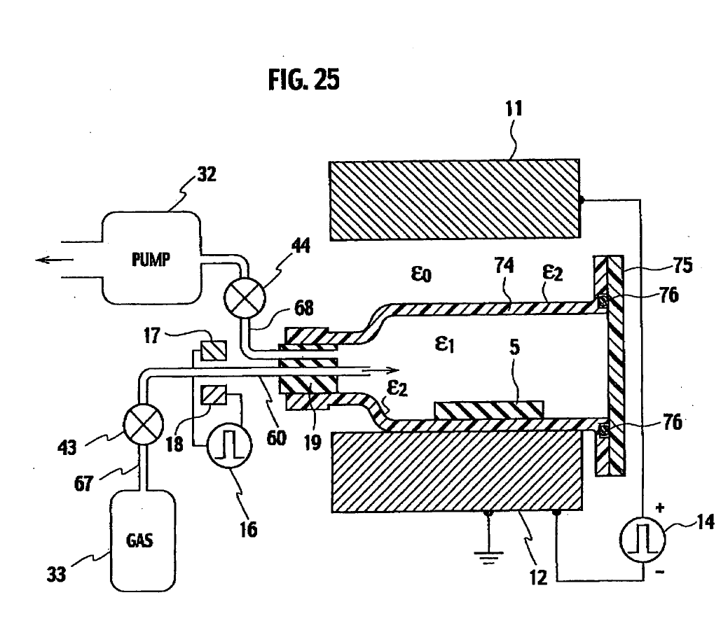

Although, in FIGS. 22-24, the dielectric housings (74, 75 and 76) are mounted on the second main electrode 12 via the second main electrode covering insulating film 77, respectively, the dielectric housing (74, 75 and 76) can be fixed directly on the second main electrode 12 as shown in FIG. 25. FIG. 25 is a cross-sectional view schematically explaining essential structure of the surface treatment apparatus in accordance with a third modification of eleventh embodiment of the present invention.

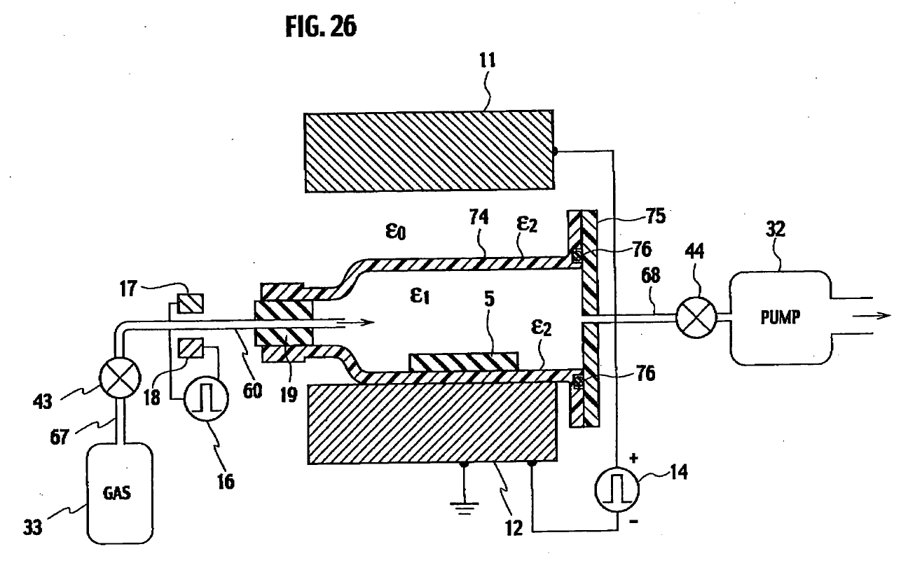

As shown in FIG. 26, a surface treatment apparatus related to a fourth modification of the eleventh embodiment of the present invention apparatus encompasses a dielectric housing (74, 75 and 76) configured to accommodate an treatment object 5; a gas introducing system (33, 67, 43, 60) (33, 67, 43, 60) configured to introduce a process gas from one end of the dielectric housing (74, 75 and 76); a vacuum evacuating system (32, 44 and 68) configured to evacuate the process gas from other end of the dielectric housing (74, 75 and 76); an excited particle supplying system (16,17 and 18) disposed at the gas supply upstream side to the dielectric housing (74, 75 and 76), configured to supply excited particles for inducing initial discharge in a main body of the dielectric housing (74, 75 and 76); and a first main electrode 11 and a second main electrode 12 disposed oppositely to each other, defining a treating region of the treatment object as a main plasma generating region disposed therebetween, wherein the excited particle supplying system (16,17 and 18) is driven at least until generation of main plasma, and main pulse of duty ratio of 10⁻⁷ to 10⁻¹ is applied between the first main electrode 11 and second main electrode 12, to generate a non-thermal equilibrium plasma flow inside the dielectric housing (74, 75 and 76), and thereby a surface of the treatment object 5 is treated.

Although, in FIG. 26, the dielectric housing (74, 75 and 76) is fixed directly on the second main electrode 12, the dielectric housings (74, 75 and 76) may be mounted on the second main electrode 12 via a second main electrode covering insulating film as shown in FIGS. 22-24.

### Twelfth Embodiment

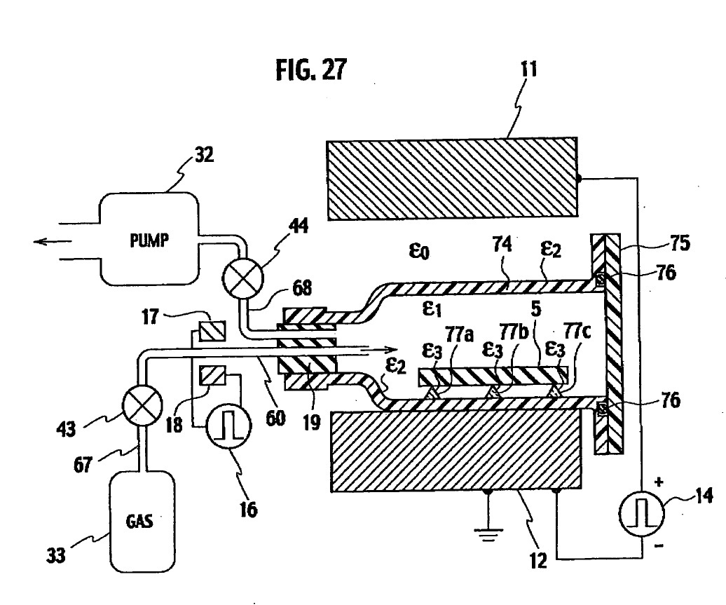

As shown in FIG. 27, a surface treatment apparatus related to a twelfth embodiment of the present invention apparatus encompasses a dielectric housing (74, 75 and 76) configured to accommodate an treatment object 5; a dielectric housing (74, 75 and 76) configured to accommodate an treatment object 5 via a plurality of protrusions 77a, 77b, 77c; a vacuum evacuating system (32,44 and 68) configured to evacuate a process gas introduced at a specific flow rate from an introducing piping provided at other end of the dielectric housing (74, 75 and 76) having one end closed, from an exhaust piping provided at the other end, and maintaining the pressure of the process gas inside the dielectric housing (74, 75 and 76) at a process pressure; an excited particle supplying system (16,17 and 18) disposed at the gas supply upstream side to the dielectric housing (74, 75 and 76), configured to supply excited particles for inducing initial discharge in a main, body of the dielectric housing (74, 75 and 76); and a first main electrode 11 and a second main electrode 12 disposed oppositely to each other, defining a treating region of the treatment object as a main plasma generating region disposed therebetween, wherein the excited particle supplying system (16,17 and 18) is driven at least until generation of main plasma, and main pulse of duty ratio of 10⁻⁷ to 10⁻¹ is applied between the first main electrode 11 and second main electrode 12, to generate a non-thermal equilibrium plasma flow inside the dielectric housing (74, 75 and 76), and thereby a surface of the treatment object 5 is treated.

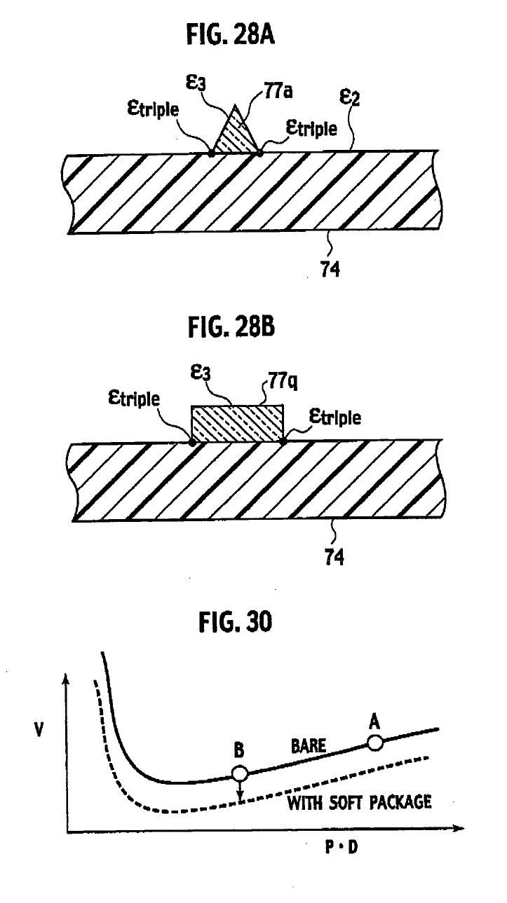

As shown in FIG. 27, the dielectric housing (74, 75 and 76) is implemented by a dielectric tube 74 and a dielectric flange plate 75, and a plurality of protrusions 77a, 77b, 77c are provided on the inner surface of the dielectric tube 74, and the treatment object 5 is mounted on the inner surface of dielectric tube 74 via protrusions 77a, 77b, 77c. If a plurality of protrusions 77a, 77b, 77c are provided on the inner surface of the dielectric tube 74, the initial voltage repaired for plasma discharge can be reduced, owing to the effect of dielectric triple point εₜᵣᵢₚₗₑ as shown in FIGS. 28A and 28B. If dielectric triple point εₜᵣᵢₚₗₑ is present in a plasma space, the plasma discharge will start from the dielectric triple point εₜᵣᵢₚₗₑ, and the initial voltage required for plasma discharge can be reduced.

### Tieteeenth Embodiment

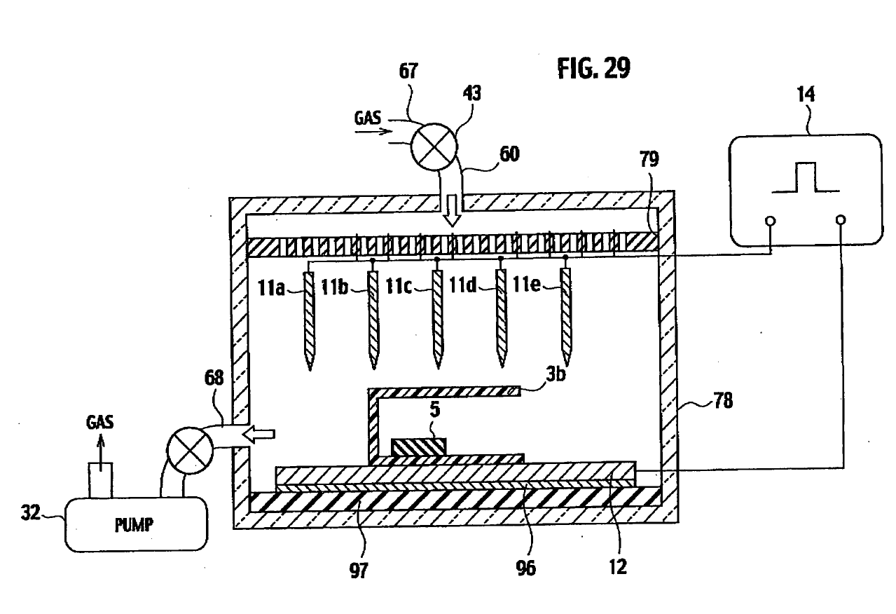

As shown in FIG. 29, a surface treatment apparatus related to a thirteenth embodiment of the present invention encompasses a process chamber 78 establishing a closed space enclosing the surrounding of the treatment object 5, which is installed in a relaxation housing 3b; a gas introducing system (67, 43, 60) for introducing a process gas from one end of the process chamber 78; a vacuum evacuating system (68, 32) for evacuating the process gas from other end of the process chamber 78; an array of first main electrodes 11a, 11b, 11c, 11d and 11e, disposed in the process chamber 78 so as to serve as an anode; a second main electrode 12 disposed in the process chamber 78 so as to serve as a cathode; and an ambient gas adjusting mechanism 79 disposed in the process chamber 78, for supplying the process gas from the array of first main electrodes 11a, 11b, 11c, 11d and 11e like a shower toward the second main electrode 12.

The relaxation housing 3b is a housing made of thin dielectric thin film. One plane of the relaxation housing 3b is made open such that ambient gas and plasma species can communicate between inside and outside of the relaxation housing 3b.

A pulse power supply 14 applies electric pulses (main pulses) across the array of first main electrodes 11a, 11b, 11c, 11d and 11e and the second main electrode 12, which implement a quasi-parallel plate electrode, so that the electric pulse can cause the fine-streamer discharge in the sealed up space, which surrounds the outside of the relaxation housing 3b. In the ambient gas adjustment mechanism 79 a plurality of gas supply holes are provided in a form of two-dimensional matrix with a predetermined pitch. The main pulse of duty ratio of 10⁻⁷ to 10⁻¹ is applied between the array of first main electrodes 11a, 11b, 11c, 11d and 11e and second main electrode 12, and the surface of the treatment object 5 is treated in non-thermal equilibrium plasma in the relaxation housing 3b.

If we assume the distance between the tip of the array of first main electrodes 11a, 11b, 11c, 11d and 11e and the top of the relaxation housing 3b is d, the film thickness of the relaxation housing 3b is t, and the inner height of the relaxation housing 3b is g, with ε₁ for the dielectric constant of process ε₂ for the dielectric constant of relaxation housing 3b, the total capacitance Cₜₒₜₐₗ of the parallel plate capacitance with area S, which is defined against the plasma space is given by:

Cₜₒₜₐₗ=S/(d/ε₀ε₁+2t/ε₀ε₂+g/ε₀ε₁) (7).

From Eq.(7), we understand that we can make electric field in the inside of the relaxation housing 3b larger than in the outside of the relaxation housing 3b, so that we can generate plasma only in the inside of the relaxation housing 3b. Namely, as shown in FIG. 30, the Paschen's curve illustrated by dotted line for the case that the relaxation housing 3b is employed will move to lower voltage side, compared to the curve illustrated by solid line for the case that the relaxation housing 3b is not employed.

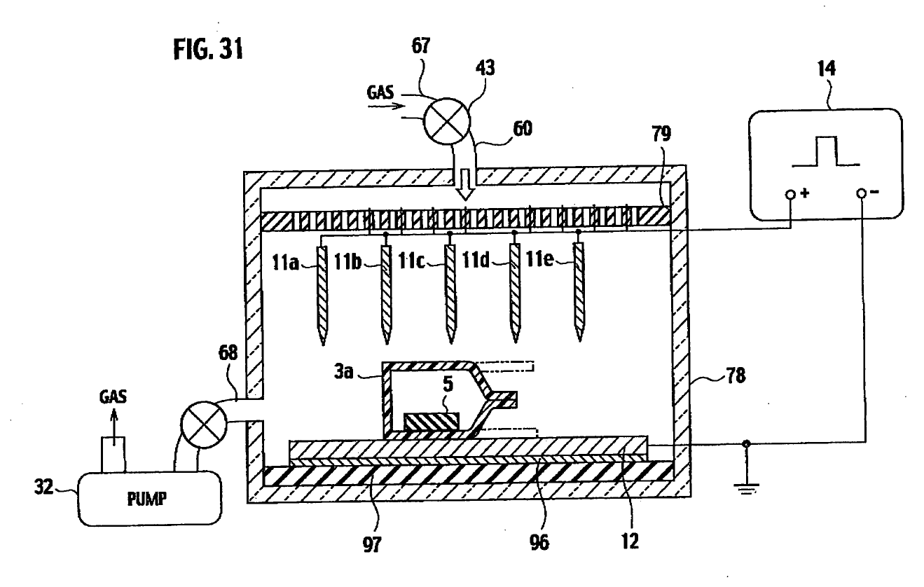

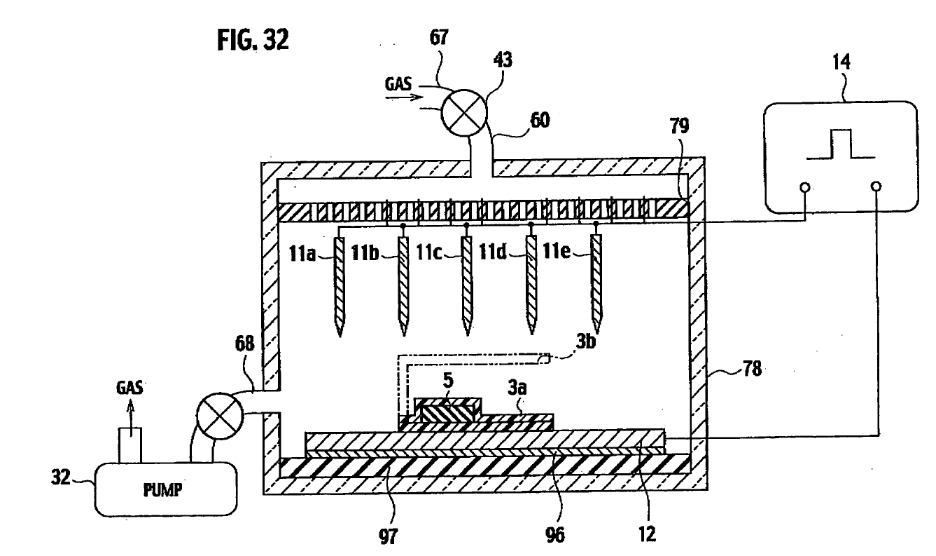

As shown in FIG. 31, when the treatment against the treatment object 5 is completed, the treatment object 5 may be hermetically sealed off by the relaxation housing 3a with inert gas such as nitrogen gas, because the relaxation housing 3a is so thin to establish a flexible behavior. Alternatively, as shown in FIG. 32, when the treatment against the treatment object 5 is completed, the treatment object 5 may be hermetically sealed off by the relaxation housing 3a with reduced pressure.

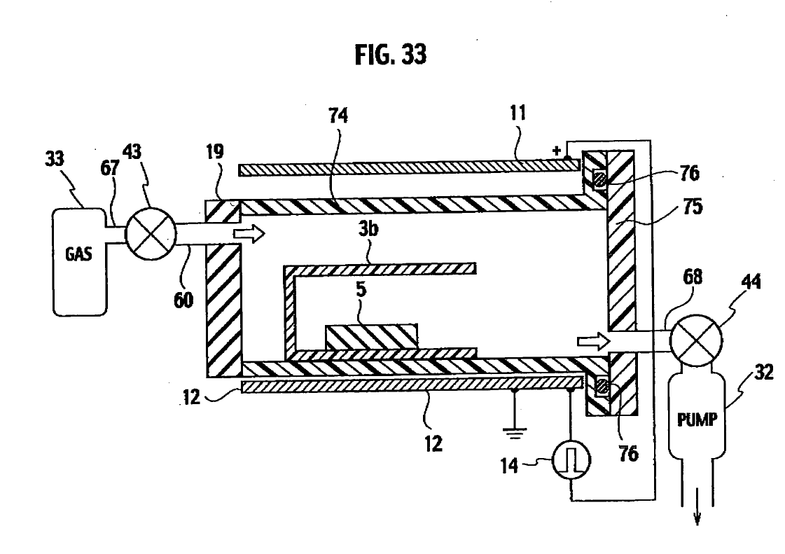

As shown in FIG. 33, a surface treatment apparatus related to a modification of the thirteenth embodiment of the present invention encompasses a process chamber (74, 75 and 76) establishing a closed space enclosing the surrounding of the treatment object 5, which is installed, in a relaxation housing 3b; a gas introducing system (33, 67, 43, 60) for introducing a process gas from one end of the process chamber (74, 75 and 76); a vacuum evacuating system (68, 44 and 32) for evacuating the process gas from other end of the process chamber (74, 75 and 76); a first main electrodes 11, disposed over the process chamber (74, 75 and 76) so as to serve as an anode; a second main electrode 12 disposed below the process chamber (74, 75 and 76) so as to serve as a cathode. The main pulse of duty ratio of 10⁻⁷ to 10⁻¹ is applied between the first main electrodes 11 and second main electrode 12, and an outer surface of the treatment object 5 is treated in non-thermal equilibrium plasma. A pulse power supply 14 applies electric pulses (main pulses) across the first main electrodes 11 and the second main electrode 12, which implement a parallel plate electrode, so that the electric pulse can cause the fine-streamer discharge in the sealed up space, which surrounds the outside of the treatment object 5.

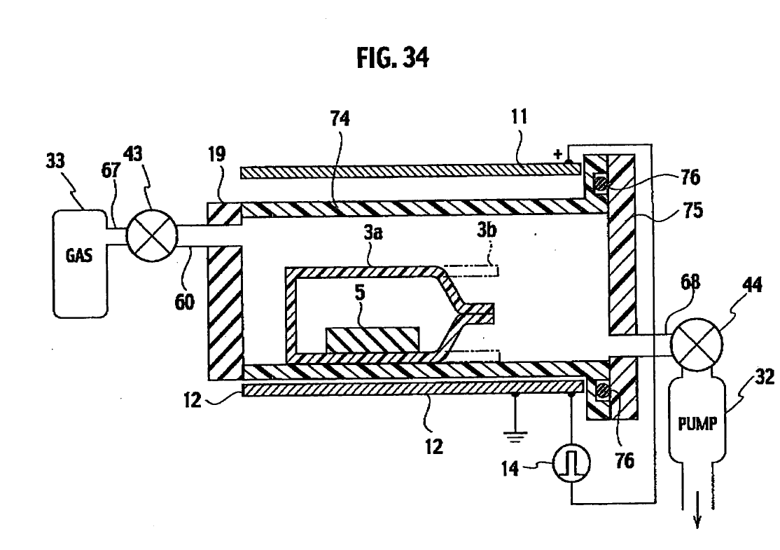

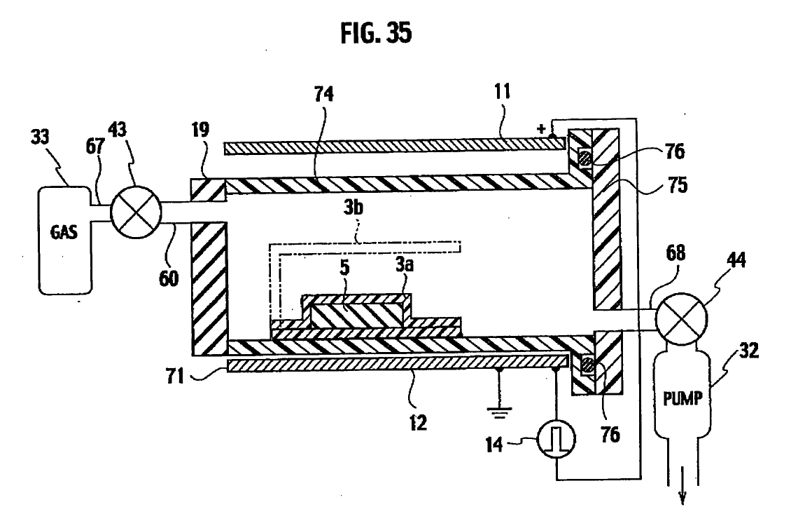

Although FIG. 33 shows the state that the treatment object 5 is under treatment by the surface treatment apparatus in accordance with the modification of the thirteenth embodiment of the present invention, as shown in FIG. 34, when the treatment against the treatment object 5 is completed, the treatment object 5 maybe hermetically sealed off by the relaxation housing 3a with inert gas such as nitrogen gas, because the relaxation housing 3a is flexible. Alternatively, as shown in FIG. 35, when the treatment against the treatment object 5 is completed, the treatment object 5 may be hermetically sealed off by the relaxation housing 3a with reduced pressure.

### Other Embodiment

Various modifications will become possible for those skilled in the art after receiving the teaching of the present disclosure without departing from the scope thereof.

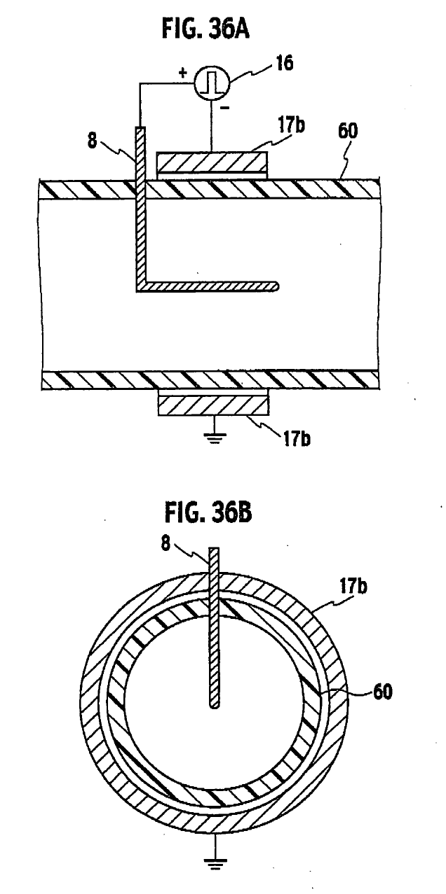

For example, each technical idea explained in first to thirteenth embodiments can be put together each other. For example, structure of the first main electrode 11c which the first modification of the second embodiment explained and the third structure of the ambient gas adjustment mechanism (62, 27, 66c) may be applied to sixth to tenth embodiments, the structure of the first main electrode 11d which described in the second modification of the second embodiment and the third structure of the ambient gas adjustment mechanism (62, 25d, 66d) may be applied to sixth to tenth embodiments. In addition, the excitation by ultraviolet rays is disclosed in the eighth embodiment, and the excitation by a plasma discharge through a parallel plate electrode is disclosed in the first to seventh and ninth to thirteenth embodiments, as an excited particle supplying system, they are disclosed as mere illustrations, and there are many other excitation mechanism of various kinds for generating initial plasma. For example, it makes go around the outside of belt-shaped (the ring which is flatness-shaped) the feed piping 60 in one electrode (the first auxiliary electrode) 17b as shown in FIGS. 36A and 36B, the other electrode (the second auxiliary electrode) 8 is done to the letter of L-shaped form, and it is established in central part of the feed piping 60, a discharged between the first auxiliary electrode 17b and the second auxiliary electrode 8.

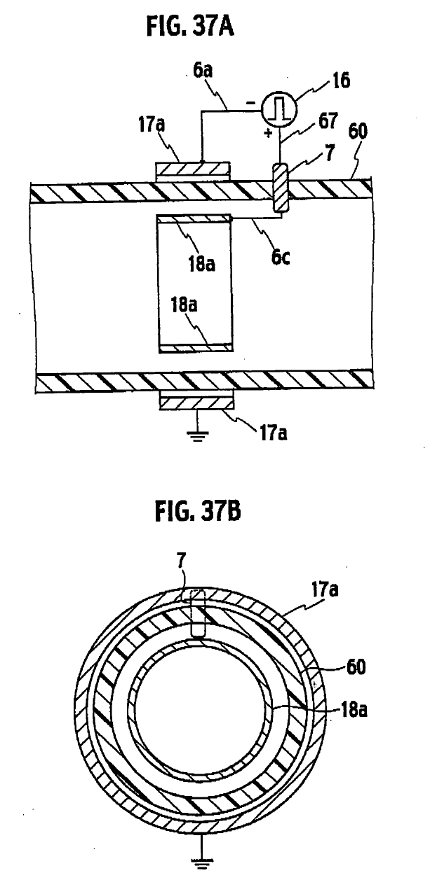

Or it makes go around the outside of belt-shaped (the ring which is flatness-shaped) the feed piping 60 in one electrode (the first auxiliary electrode) 17a as shown in FIGS. 37A and 37B, in other electrode (the second auxiliary electrode) 18a, belt-shaped (the ring which is flatness-shaped), and inside of the feed piping 60 is gone around, it is discharged between the first auxiliary electrode 17a and the second auxiliary electrode 18a.

In FIGS. 37A and 37B, electric current introduction terminal (feedthrough) 7 auxiliary pulse power supply 16 to a method of but it is excited in a case supplied in electrode (the second auxiliary electrode) 18a, voltage supply is not limited in an illustration of FIGS. 37A and 37B. Outside interconnection 67 is connected to electric current introduction terminal (feedthrough) 7 by supporting pulse power supply 16, the is connected to electric current introduction terminal (feedthrough) 7 and other electrode (the second auxiliary electrode) 18a in the inside interconnection 6c. In addition, it is connected to supporting pulse power supply 16 and one electrode (the first auxiliary electrode) 17a in outside interconnection 17a. In addition, excitation by ultraviolet rays with the use of a multiplex reflection was explained in the eighth embodiment, it is not necessary to always use a multiplex reflection, and, by mechanism to make an introduction direction of the process gas run one ultraviolet rays beam, an excitation particle can be generated.

In addition, an excitation particle may be generated by mechanism of radioactive rays aside from ultraviolet rays, radioactive rays by synchrotron radiation, for example.

In addition, treatment object illustrated one case in first to thirteenth embodiments, if it is confronted each other, and the first main electrode 11b and the second main electrode 12 are disposed to catch all several treatment object, the treatment object of a plural number can be processed simultaneously.

In this case if inside of plural treatment object is processed, it being necessary valves accompanying injecting piping of the process gas as opposed to each treatment object (introduction piping) and exhausting piping, of course.

Thus, the present invention of course includes various embodiments and modifications and the like which are not detailed above. Therefore, the scope of the present invention will be defined in the following claims.

What is claimed is:

1. A surface treatment apparatus comprising:
a gas introducing system configured to introduce a process gas from one end of a tubular treatment object;
a vacuum evacuating system configured to evacuate the process gas from other end of the treatment object;
an excited particle supplying system disposed at the gas supply upstream side to the treatment object, configured to supply excited particles for injuring initial discharge in a main body of the treatment object; and
a first main electrode and a second main electrode disposed oppositely to each other, defining a treating region of the treatment object as a main plasma generating region disposed therebetween,
wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the treatment object, and thereby an inner surface of the treatment object is treated.

2. The surface treatment apparatus according to claim 1, further comprising:
a process chamber establishing a closed space enclosing the surrounding of the treatment object; and
an ambient gas adjusting mechanism, having the first main electrode as the anode and the second main electrode as the cathode, configured to supply the process gas in the process chamber, from the first main electrode like a shower toward the second main electrode, and evacuating the shower of the process gas from a part of the process chamber,
wherein the main pulse is applied between the first main electrode and second main electrode, and an outer surface of the treatment object is further treated in non-thermal equilibrium plasma.

3. The surface treatment apparatus according to claim 1, wherein a half width of pulse width of the main pulse is 10 to 500 ns,
the pulse width is set according to an interval of the anode and cathode, and such that the pulse voltage application is completed before an arc discharge current begins to flow in the plasma generation between the anode and cathode, the plasma generation lapses from a glow discharge, through a streamer discharge to the arc discharge.

4. The surface treatment apparatus according to claim 2, wherein the ambient gas adjusting mechanism has a second vacuum evacuating system configured to evacuate the space enclosing the surrounding of the treatment object.

5. The surface treatment apparatus according to claim 1, wherein the excited particle supplying system is any one of ultraviolet ray generator, laser beam generator, electron beam generator, radiation generator, and high temperature heater.

6. The surface treatment apparatus according to claim 1, wherein discharge of the non-thermal equilibrium plasma is fine streamer discharge.

7. The surface treatment apparatus according to claim 1, wherein discharge of the non-thermal equilibrium plasma has a maximum rise rate dV/dt of voltage of the main pulse, which is applied between the first main electrode and the second main electrode, in a range of 10 kV/μs to 1000 kV/μs.

8. A surface treatment apparatus comprising:
a vacuum evacuating system configured to evacuate a process gas introduced at a specific flow rate from an introducing piping provided at other end of a tubular treatment object having one end closed, from an exhaust piping provided at the other end, and maintaining the pressure of the process gas inside the treatment object at a process pressure;
an excited particle supplying system disposed at the gas supply upstream side to the treatment object, configured to supply excited particles for inducing initial discharge in a main body of the treatment object; and
a first main electrode and a second main electrode disposed oppositely to each other, defining a treating region of the treatment object as a main plasma generating region disposed therebetween,
wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the treatment object, and thereby an inner surface of the treatment object is treated.

9. The surface treatment apparatus according to claim 8, wherein, a half width of pulse width of the main pulse is 10 to 500 ns,
the pulse width is set according to an interval of the anode and cathode, and such that the pulse voltage application is completed before an arc discharge current begins to flow in the plasma generation between the anode and cathode, the plasma generation lapses from a glow discharge, through a streamer discharge to the arc discharge.

10. A surface treatment apparatus comprising:
a vacuum manifold unit connected to other end of a tubular treatment object having one end closed, for sealing process gas at specified pressure inside of the treatment object from the other end;
an excited particle supplying system disposed at the other end side, configured to supply excited particles for inducing initial discharge in a main body of the treatment object; and
a first main electrode and a second main electorate disposed oppositely to each other, defining a treating region of the treatment object as a main plasma generating region disposed therebetween,
wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the treatment object, and thereby an inner surface of the treatment object is treated.

11. The surface treatment apparatus according to claim 10, wherein a half width of pulse width of the main pulse is 10 to 500 ns,
the pulse width is set according to an interval of the anode and cathode, and such that the pulse voltage application is completed before an arc discharge current begins to flow in the plasma generation between the anode and cathode, the plasma generation lapses from a glow discharge, through a streamer discharge to the arc discharge.

12. A surface treatment apparatus comprising:
a vacuum evacuating system configured to generate a gas flow by evacuating a process gas introduced from one end of a tubular trunk pipe of a treatment object, the treatment object having the tubular trunk pipe and a branch pipe branched off from the trunk pipe, from the other end of the trunk pipe and an end portion of the branch pipe;
an excited particle supplying system disposed at the gas supply upstream side to the treatment object, configured to supply exerted particles for inducing initial discharge in a main body of the treatment object; and
a first main electrode and a second main electrode disposed oppositely to each other, defining a treating region of the treatment object as a main plasma generating region disposed therebetween,
wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the treatment object, and thereby an inner surface of the treatment object is treated.

13. The surface treatment apparatus according to claim 12, wherein a half width of pulse width of the main pulse is 10 to 500 ns,
the pulse width is set according to an interval of the anode and cathode, and such that the pulse voltage application is completed before an arc discharge current begins to flow in the plasma generation between the anode and cathode, the plasma generation lapses from a glow discharge, through a streamer discharge to the arc discharge.

14. A surface treatment apparatus comprising:
a vacuum evacuating system configured to generate a gas flow by evacuating a process gas introduced from one end of a tubular trunk pipe of a treatment object and end portion of a branch pipe of the treatment object, the treatment object having the tubular trunk pipe arid the branch pipe branched off from the trunk pipe, from the other end of the trunk pipe;
an excited particle supplying system disposed at the gas supply upstream side to the treatment object, configured to supply excited particles for inducing initial discharge in a main body of the treatment object; and
a first main electrode and a second main electrode disposed oppositely to each other, defining a treating region of the treatment object as a main plasma generating region disposed therebetween,
wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the treatment object, and thereby an inner surface of the treatment object is treated.

15. The surface treatment apparatus according to claim 14, wherein a half width of pulse width of the main pulse is 10 to 500 ns,
the pulse width is set according to an interval of the anode and cathode, and such that the pulse voltage application is completed before an arc discharge current begins to flow in the plasma generation between the anode and cathode, the plasma generation lapses from a glow discharge, through a streamer discharge to the arc discharge.

16. A surface treatment apparatus comprising:
an excited particle supplying system disposed at the gas supply upstream side of a tubular treatment object made of dielectric material the treatment object having a length greater than the diameter, configured to supply excited particles for inducing initial discharge in a main body of the treatment object; and
a first main electrode and a second main electrode disposed oppositely to each other, defining a treating region of the treatment object as a main plasma generating region disposed therebetween,
wherein a process gas is introduced from one end of the treatment object to form a gas flow inside of the treatment object, and the pressure of the gas flow is adjusted to a process pressure in a range of 20 kPa to 100 kPa, the excited particle supplying system is driven at least until generation of main plasma, and main pulse of duty ratio of 10⁻⁷ to 10⁻¹ is applied between the first main electrode and second main electrode to generate a non-thermal equilibrium plasma flow inside the treatment object, and thereby an inner surface of the treatment object is treated.

17. The surface treatment apparatus according to claim 16, wherein a half width of pulse width of the main pulse is 10 to 500 ns,
the pulse width is set according to an interval of the anode and cathode, and such that the pulse voltage application is completed before an arc discharge current begins to flow in the plasma generation between the anode and cathode, the plasma generation lapses from a glow discharge, through a streamer discharge to the arc discharge.

18. A surface treatment apparatus comprising:
a dielectric housing configured to accommodate an treatment object;
a gas introducing system configured to introduce a process gas from one end of the dielectric housing;
a vacuum evacuating system, configured to evacuate the process gas from other end of the dielectric housing;
an excited particle supplying system disposed at the gas supply upstream side to the dielectric housing, configured to supply excited particles for inducing initial discharge in a main body of the dielectric housing; and
a first main electrode and a second main electrode disposed oppositely to each other, defining a treating region of the treatment object as a main plasma generating region disposed therebetween,
wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the dielectric housing, and thereby a surface of the treatment object is treated.

19. The surface treatment apparatus according to claim 18, wherein a half width of pulse width of the main pulse is 10 to 500 ns,
the pulse width is set according to an interval of the anode and cathode, and such that the pulse voltage application is completed before an arc discharge current begins to flow in the plasma generation between the anode and cathode, the plasma generation lapses from a glow discharge, through a streamer discharge to the arc discharge.

20. A surface treatment apparatus comprising:
a dielectric housing configured to accommodate an treatment object;
a vacuum evacuating system configured to evacuate a process gas introduced at a specific flow rate from an introducing piping provided at other end of the dielectric housing having one end closed, from an exhaust piping provided at the other end, and maintaining the pressure of the process gas inside the dielectric housing at a process pressure;
an excited particle supplying system disposed at the gas supply upstream side to the dielectric housing, configured to supply excited particles for inducing initial discharge in a main body of the dielectric housing; and
a first main electrode and a second main electrode disposed oppositely to each other, defining a treating region of the treatment object as a main plasma generating region disposed therebetween,
wherein the excited particle supplying system is driven at least until generation of main plasma, and main pulse of duty ratio of 10⁻⁷ to 10⁻¹ is applied between the first main electrode and second main electrode, to generate a non-thermal equilibrium plasma flow inside the dielectric housing, and thereby a surface of the treatment object is treated.

21. The surface treatment apparatus according to claim 20, wherein, a half width of pulse width of the main pulse is 10 to 500 ns,
the pulse width is set according to an interval of the anode and cathode, and such that the pulse voltage application is completed before an arc discharge current begins to flow in the plasma generation between the anode and cathode, the plasma generation lapses from a glow discharge, through a streamer discharge to the arc discharge.
